(19) United States
(12) Patent Application Publication
(10) Pub. No.: US 2020/0347432 A1
(43) Pub. Date: Nov. 5, 2020

(54) RAPID METHODS FOR DETERMINING MICROORGANISM GROWTH IN SAMPLES OF HUMAN ORIGIN

(71) Applicant: SELUX DIAGNOSTICS, INC., Charlestown, MA (US)

(72) Inventors: Kristin Baker, Charlestown, MA (US); Kelly Flentie, Charlestown, MA (US); Aleksandar Vacic, Charlestown, MA (US); Eric Stern, Charlestown, MA (US); Ali Ersen, Charlestown, MA (US); Benjamin R. Spears, Charlestown, MA (US); Matthew Briscoe, Charlestown, MA (US)

(21) Appl. No.: 16/863,603

(22) Filed: Apr. 30, 2020

Related U.S. Application Data
(60) Provisional application No. 62/876,147, filed on Jul. 19, 2019.
(60) Provisional application No. 62/840,657, filed on Apr. 30, 2019.
(60) Provisional application No. 62/858,846, filed on Jun. 7, 2019.
(60) Provisional application No. 62/946,023, filed on Dec. 10, 2019.
(60) Provisional application No. 62/970,632, filed on Feb. 5, 2020.

(51) Int. Cl. C12Q 1/06; G01N 1/28; G01N 1/40; G01N 25/48; G01N 21/31; G01N 21/64; G01N 15/14

(52) U.S. Cl. CPC C12Q 1/06; G01N 1/28; G01N 1/4077; G01N 25/48; G01N 2015/0073; G01N 21/6486; G01N 15/14; G01N 2001/4083; G01N 21/31

(57) ABSTRACT

Continuous monitoring of blood cultures using pH- (or CO₂—) based detection platforms is the current clinical gold standard. Despite the ubiquity of these systems in state-of-the-art clinical microbiology laboratories, they offer slow times-to-result (TTR) because microorganism detection typically requires >10⁹ colony forming units (CFU) to be present whereas only 1-1000 CFU are typically present in septic patient blood samples. These TTRs are further lengthened for samples collected from spoke sites in consolidated hub-and-spoke laboratory models, an increasingly common model for integrated hospital networks and reference laboratories, because sample transport time, typically >4 hours, is lost. Here we introduce new methods that allow microorganisms to be detected at <10⁵ CFU and that enable sample incubation during courier transport from spoke collection sites to the central laboratory hub.

### CROSS-REFERENCE TO RELATED APPLICATIONS

This application claims the benefit of priority to U.S. Provisional Application Ser. No. 62/876,147, filed Jul. 19, 2019; U.S. Provisional Application Ser. No. 62/840,657, filed Apr. 30, 2019; U.S. Provisional Application Ser. No. 62/858,846, filed Jun. 7, 2019; U.S. Provisional Application Ser. No. 62/946,023, filed Dec. 10, 2019; and U.S. Provisional Application Ser. No. 62/970,632, filed Feb. 5, 2020. Each of these applications are expressly incorporated by reference herein in their entirety and for all purposes.

### FIELD OF THE DISCLOSURE

This application relates to clinical microbiology systems and methods, particularly for the preparation and analysis of blood- and blood-derived samples.

### BACKGROUND

The current clinical microbiology laboratory state-of-the-art is continuous monitoring of blood cultures using platforms similar to that described in U.S. Pat. No. 5,624,814 . These platforms gained favor over manual methods because of improved reproducibility, primarily the minimization of contamination, and human usability. This usability is of particular importance because typically no more than 10% of blood cultures are positive for microorganism growth, and also because they often registered more positive cultures than did manual methods.

Continuous monitoring blood culture systems suffer three primary shortcomings that may delay results. First, these systems require bacteria to grow in the presence of bloodborne agents that may inhibit bacterial growth, such as white blood cells and platelets, cationic peptides, and intravenous antibiotics. Second, because of the CO₂-based detection mechanisms employed by the leading systems, positive results are often not obtained until bacterial levels of 10⁷-10⁸ CFU/mL are achieved. Third, since continuous monitoring is required by the detection modalities, in a consolidated healthcare system where bottles can be collected at sites that are geographically separated from the central clinical laboratory, incubation of the bottles cannot commence until they are loaded into the central laboratory's continuous monitoring platform. Thus, time waiting and in transport may be lost, as a plurality of culture growth time must be in the system. In addition to being a significant disadvantage for integrated health networks, the inability to incubate samples outside the laboratory may also slow results in large hospitals, where bottles may not be immediately transported to laboratories upon being filled.

Continuous monitoring blood culture systems have remained the gold standard since the mid-1990s for two primary reasons: 1) cost and 2) positive predictive values (PPVs). The current clinical microbiology laboratory paradigm whereby automated blood culture is performed on every sample is a very effective cost filter. Such processing requires near-zero medical technologist time and effort and typically screens out >90% of samples. Laboratories can then focus their effort and budgets on the positive samples. Furthermore, the cost of blood bottles is low, thus not only do these systems effectively screen out most samples but they do so with a low-cost consumable.

Perhaps the most critical parameter for a blood culture system is its PPV. A positive blood culture determination not only provides confirmatory information about an infection to physicians but is also necessary for downstream diagnostic testing, including Gram staining, microorganism identification, and antimicrobial susceptibility testing (and optional resistance testing). Today's continuous monitoring systems—the BACTEC® (Becton-Dickinson), the BacT/Alert™ (bioMerieux), and the VersaTrek™ (Thermo Fisher)—detect microorganism growth from an entire 8-10 cc sample and utilize resins or dilution factors to minimize potential inhibitory effects of native and exogenous antimicrobials present in blood.

The importance of collecting at least 8-10 cc of blood from adults for culturing has been demonstrated in multiple publications, such as Detection of bloodstream infections in adults: how many blood cultures are needed? (Lee A, Mirrett S, Reller L B, and Weinstein P M. J Clin Microbiol 45:3546-3458, 2007). This is due to the fact that bacteremic patients may have very few viable organisms circulating in their blood. In order for a new blood culture system to maintain a PPV equivalent to that of today's systems, it is therefore imperative to enable collections of 8-10 cc blood per receptacle.

### SUMMARY

This disclosure provides systems and methods for microbiological blood sample collection, processing and analysis that may reduce times required to achieve positive blood culture determinations.

In one aspect, this disclosure describes a method of interrogating a blood sample for microorganism. This method may comprise separating the blood sample into a first fraction and a second fraction under conditions that tend to concentrate microbes into the first fraction, incubating the first and second fractions under conditions suitable for microbial growth, and interrogating the first and second fractions for microbial growth, wherein (a) the first fraction is interrogated using a first growth detection method for a first time interval and the second fraction is interrogated using a second growth detection method during a second time interval (b) the first growth detection method is more sensitive to microbial growth than the second detection method, (c) the second time interval is longer than the first time interval, and (d) if growth is not detected in the first fraction using the first growth detection method during the first time interval, the first fraction is interrogated for microbial growth for the remainder of the second time interval using a growth detection method other than the first growth detection method.

In various embodiments, the growth detection method other than the first growth detection method may be the second growth detection method. The first growth method may be calorimetry. The second growth detection method may be pH, gaseous, or optical and, if optical, may be selected from the group consisting of: optical measurement of turbidity; optical measurement of absorbance at one or more wavelengths, optical detection of a signal of a metabolic indicator dye; monitoring of autofluorescence, flow cytometry or any combination of the foregoing. The first and second fractions may comprise microbial suspensions. The step of separating the blood sample into first and second fractions may comprise centrifugation. The blood sample may be incubated under conditions favorable for microbial growth prior to the step of separating the blood sample into a first fraction and a second fraction, and the conditions favorable for microbial growth optionally comprise one or more of a temperature above ambient temperature, temperature of about 37 degrees centigrade, addition of a nutrient or a nutrient media, and/or addition of a material that adsorbs or inactivates an antimicrobial in the blood sample. Microbial growth may not be monitored during the incubation preceding the separation step.

In an aspect, this disclosure describes a method of interrogating a blood sample for microorganisms. This method may comprise contacting the blood sample with a resin capable of adsorbing antimicrobial agents, performing one or more concentration steps to concentrate microorganisms into: a) a pellet and b) a supernatant, introducing a first subsample comprising at least a portion of the pellet into a calorimeter, measuring heat flow from the first subsample, thereby monitoring growth of the first subsample, and retaining a second subsample comprising a portion of the supernatant, wherein the second subsample is monitored for growth (i) by a method other than calorimetry, and/or (ii) over a time interval longer than an interval of monitoring the first subsample.

In various embodiments, the method may include that if no growth is measured, the first subsample may be removed from the calorimeter after a pre-determined period of time of about 0.5, 1, 2, 3, 4, 5 days. The second subsample may be monitored for growth by optical, pH, gaseous, or impedance methods. The growth of at least one of the first and second subsamples may be monitored based on an absolute signal. The growth of at least one of the first and second subsamples may be monitored based on a relative signal. The second sample may further comprise at least a portion of the pellet. The retained supernatant and remainder of the pellet may be monitored for growth by optical, pH, gaseous, or impedance methods. The blood sample may be incubated under conditions promoting microorganism growth prior to the concentration step. The calorimeter may be a differential scanning or isothermal calorimeter. The first and second subsamples may be monitored for growth in parallel for a first interval, and wherein if growth is not detected in the first sample during the first interval, the first subsample is removed from the calorimeter and is monitored for growth by a method other than calorimetry for a remainder of the time interval over which the second subsample is monitored for growth. The supernatant may not undergo substantial concentration or purification. All or substantially all of a volume of the supernatant may be included in the second subsample. The blood sample may be collected in a collection vessel, the concentration steps comprise centrifugation of the blood sample in the collection vessel, and the supernatant is aspirated or decanted from the collection vessel following centrifugation and optionally returned to the collection vessel following removal of the pellet. The step of retaining the second subsample may comprise retaining the supernatant in the collection vessel. The blood sample may be incubated under conditions favorable for microbial growth prior to the one or more concentration steps, and wherein the conditions favorable for microbial growth optionally comprise one or more of a temperature above ambient temperature, temperature of about 37 degrees centigrade, addition of a nutrient or a nutrient media, and/or addition of a material that adsorbs or inactivates an antimicrobial in the blood sample. Microbial growth may not be monitored during the incubation preceding the one or more concentration steps.

In an aspect, this disclosure describes a method of detecting microbial growth in a blood sample. This method may comprise the steps of causing one or more endothermic processes in the blood sample and detecting a heat flow from the sample, wherein the heat flow has a non-negative slope, thereby detecting microbial growth.

In various embodiments, the method may include that the endothermic processes are caused by one or more of an anticoagulant and a lytic agent applied to the blood sample. The blood sample may be pre-incubated under conditions that favor microbial growth prior to the step of detecting the heat flow from the sample. Detection of heat flow may comprise isothermal calorimetry. The endothermic processes may comprise a micellization reaction. The step of causing the endothermic process may include contacting the blood sample with a lytic reagent, optionally saponin. The step of causing the endothermic process may include contacting the blood sample with an anticoagulant, optionally sodium polyanethole sulfonate.

In one aspect, the disclosure relates to methods of interrogating a blood sample for microorganisms (e.g., for the presence of, or the growth of, microorganisms). Generally, methods according to this aspect of the disclosure involve contacting a blood sample with a resin capable of adsorbing antimicrobial agents (e.g., antimicrobial agents present in the blood at the time it is drawn), performing one or more concentration steps to concentrate the microorganisms into (a) a pellet (or other concentrated fraction) and (b) a residual supernatant (or comparatively depleted fraction), introducing at least a portion of the pellet into an isothermal calorimeter (optionally held at 31-40° C.) and measuring heat flow therefrom, whereby microorganism growth (and, implicitly, microorganism presence or absence) is determined based on an absolute or relative heat signal. At least a portion of the supernatant is also retained as a “backup” sample, which is also monitored for growth. In some cases, if no microbial growth is detected from the pellet/concentrated fraction of the blood sample by calorimetery after a given interval (which may be fixed or variable, for instance about 0.5, 1, 2, 3, 4, 5 days), the pellet or concentrated fraction is removed from the calorimeter and transferred to one or more other systems for determining growth (referred to as “secondary” growth determining systems, which may utilize, without limitation, optical, pH, gaseous, or impedance methods for the detection of microbial presence or growth). Blood samples used in embodiments according to this aspect of the disclosure may be 8m1 or more in volume, and/or may be collected in a receptacle such as a blood culture bottle. The blood sample may, alternatively or additionally, be contacted with one or more of an anticoagulant (e.g., one or more of sodium polyanethole sulfonate (SPS) and/or citrate), a nutrient or a nutrient media, and/or a lytic agent capable of selectively lysing mammalian cells (such as saponin or other agents known in the art or described herein). The concentrated fraction/pellet, in the form in which it is placed in the calorimeter, may have any suitable volume, including without limitation 0.5, 0.7, 1, 2, 3, 4, 5, 6, 7, 8, 9, 10 or more mL. In some cases, at least one additional blood sample drawn from the same patient is interrogated for microbial growth in parallel using direct pH, gaseous, or optical methods. In some cases, the adsorptive resin is isolated from the liquid reagents in the receptacle prior to the addition of blood sample to the receptacle; alternatively, or additionally, the lytic agent (e.g., saponin) is isolated from the resins, nutrient media, and anticoagulant prior to the addition of blood sample to the receptacle; or the resin(s) are isolated from the anticoagulant and/or the nutrient media prior to the addition of blood sample. The resin can also be magnetic and/or supported by a solid substrate.

Continuing with this aspect of the disclosure, the receptacle can be under negative pressure such that the blood sample fills the receptacle when connected with a standard fitting to a venous IV, and/or can contain a gas mixture optimized for aerobic growth. Where nutrient media is used, the nutrient media can be any suitable media including without limitation tryptic soy broth. In some embodiments, the blood sample is incubated under conditions promoting microorganism growth prior to the concentration step; for clarity, in these instances, the incubation conditions are distinct from the conditions of the lysis and concentration steps. This incubation can be performed, in some cases, in a portable sample incubation system. Following fractionation or separation of the blood sample into concentrated (e.g., pellet) and dilute (or depleted, such as a supernatant) fractions (which separation can be achieved by any suitable means, including without limitation by centrifugation, filtration, flocculation, and/or magnetic separation), the concentrated fraction is again incubated under conditions that promote microbial growth. Where separation of concentrated and depleted or dilute fractions is achieved by centrifugation, it is performed at relative centripetal force (RCF) values of 1,000×g-50,000×g or 1,000×g-10,000×g. The resulting concentrated fraction/pellet can be introduced into the calorimeter at a volume of ≤20, ≤10, ≤8, ≤6, ≤4, ≤2, ≤1, or ≤0.5 ml. In some cases, fresh nutrient media and/or agar are introduced into the concentrated fraction prior to introduction into the calorimeter. Calorimetry is generally, but not necessarily, performed in a dedicated vessel or receptacle rather than the vessel or receptacle used for collection and/or processing of the blood sample. Any suitable calorimetry mode may be used to detect microbial growth, including without limitation differential scanning calorimetery and isothermal calorimetry, and microbial growth can be assessed by measuring relative and/or absolute heat flows from the sample. In some cases, the choice of calorimetric mode and/or measurement of relative vs. absolute heat flows is made based on a characteristic of the patient sample or the concentrated fraction. For instance, if a sample has been subjected to pre-incubation after collection but before processing for an interval of, e.g., 3, 4, 5, 6, 7, 8, 9, or 10 or more hours, heat flow may be determined in a different manner than for samples below this threshold. Samples can be loaded into, or removed from, the calorimeter on an individual or on demand basis, or they may be added and/or removed in batches, either when samples have tested positive or when they have not tested positive after a given interval.

Staying with this aspect of the disclosure, if growth is not detected after 12, 24, 48, 60, 72 or more hours in the calorimeter, the concentrated fraction can be removed from the calorimeter and placed in the secondary growth detection system (e.g., the same system that is used to interrogate the depleted/dilute/supernatant fraction). Optionally, the total time that the concentrated fraction is interrogated in both the calorimeter and the secondary growth detection system can be about 4, 5, 6 or more days. The secondary growth determination systems, which can be used for both supernatant and post-calorimetry pellet samples, can utilize any number of detection modes, which are described herein. These modes include, without limitation, optical absorbance, optical spectroscopy, optical microscopy, pH measurement, gaseous measurement, mass measurement, or electrical measurement. In some cases, both the concentrated fraction/pellet and the depleted fraction/supernatant are interrogated in parallel, and the result of the growth measurement in one or both of the fractions can be used as the basis for a determination of a microbe-positive sample. After the sample is called positive, other draws from the same patient, or any reserved portion of the original sample can be used for , e.g., microbial identification and/or antimicrobial susceptibility testing.

In another aspect, this disclosure relates to lysis and/or centrifugation methods for preparing concentrated (also referred to as pellet) and depleted/dilute (also referred to as supernatant) fractions. In various embodiments according to this aspect, centrifugation of whole or lysed blood or another blood-derived fluid is performed in a vessel having a density layer or gradient that retards or prevents the in-migration of certain species while allowing other species to pass. In some cases, the density layer comprises a “cushioning fluid” that is a water -miscible or -immiscible fluid having a density greater than that of some, most, or all prokaryotic and/or eukaryotic cells. The cushioning fluid can be added directly prior to the concentration step in some cases, or it may be added simultaneously with the blood sample, or it may be present in the vessel into which the blood sample is collected.

In yet another aspect, this disclosure relates to methods for culturing blood samples suspected of comprising microorganisms. These methods generally involve contacting a blood sample or a blood-derived sample (e.g., a sample taken directly from a patient or subject, or a sample that has been subjected to one or more processing steps) with a material (e.g., a resin) capable of adsorbing or inactivating an antimicrobial agent present in the blood. A concentration step separates the blood into first and second fractions that are microbe-enriched and microbe-depleted. In some cases, these fractions are generated by centrifugation, and comprise a pellet and a supernatant, respectively. At least a portion of the first fraction is measured for microorganism growth, while the second fraction is retained as a “backup” sample; the backup sample may also comprise a portion of the first sample that is not used for other purposes. In certain embodiments, the first fraction is monitored using an optical growth measurement, though in other embodiments the monitoring is done by calorimetry (e.g., isothermal or differential scanning calorimetry or any other suitable calorimetry mode). The blood sample can comprise at least 8 ml in volume and is optionally collected in a dedicated receptacle (e.g., a blood culture bottle). In some cases, the blood sample is contacted with a nutrient or nutrient media, an anticoagulant and/or a lytic agent (e.g., saponin) prior to the concentration step. The first fraction may have any suitable volume, e.g., ≤20, ≤10, ≤8, ≤6, ≤4, ≤2, ≤1, or ≤0.5 ml. While the first fraction (or the backup sample) is measured for microorganism growth, it can be held at a stable temperature, e.g., between 31° and 40° Centigrade. Similarly, the backup sample can be monitored for microbial growth using, e.g., by optical (scattering or absorbance), pH, gaseous, or impedance methods. In various instances, the pre-incubation components, which include they lytic agent, the antimicrobial adsorptive material, nutrient media and the anticoagulant are mixed together before the blood sample is added, though in some cases it may be desirable to keep one or more components separate from the others prior until the blood sample is collected. As is discussed above in connection with other aspects of this disclosure, the vessel into which the blood sample is collected may be under negative pressure, and/or may comprise a gas mixture optimized for aerobic microorganism growth. Where nutrient media is used, the nutrient media can be any suitable media including without limitation tryptic soy broth. In some embodiments, the blood sample is incubated under conditions promoting microorganism growth prior to the concentration step; for clarity, in these instances, the incubation conditions are distinct from the conditions of the lysis and concentration steps. This incubation can be performed, in some cases, in a portable sample incubation system. Also as discussed above in connection with other aspects of this disclosure, the blood sample can be incubated under conditions promoting microorganism growth prior to the concentration step; for clarity, in these instances, the incubation conditions are distinct from the conditions of the lysis and concentration steps. The concentration step can comprise any suitable concentration method, including without limitation by centrifugation, filtration, flocculation, and/or magnetic separation), the concentrated fraction is again incubated under conditions that promote microbial growth. Where separation of concentrated and depleted or dilute fractions is achieved by centrifugation, it is performed at relative centripetal force (RCF) values of 1,000×g-50,000×g or 1,000×g-10,000×g. The resulting concentrated fraction/pellet can be introduced into the calorimeter at a volume of ≤20, ≤10, ≤8, ≤6, ≤4, ≤2, ≤1, or ≤0.5 ml. In some cases, fresh nutrient media and/or agar are introduced into the concentrated fraction prior to introduction into the calorimeter. Calorimetry is generally, but not necessarily, performed in a dedicated vessel or receptacle rather than the vessel or receptacle used for collection and/or processing of the blood sample. Any suitable calorimetry mode may be used to detect microbial growth, including without limitation differential scanning calorimetery and isothermal calorimetry, and microbial growth can be assessed by measuring relative and/or absolute heat flows from the sample. In some cases, the choice of calorimetric mode and/or measurement of relative vs. absolute heat flows is made based on a characteristic of the patient sample or the concentrated fraction. For instance, if a sample has been subjected to pre-incubation after collection but before processing for an interval of, e.g., 3, 4, 5, 6, 7, 8, 9, or 10 or more hours, heat flow may be determined in a different manner than for samples below this threshold. Samples can be loaded into, or removed from, the calorimeter on an individual or on demand basis, or they may be added and/or removed in batches, either when samples have tested positive or when they have not tested positive after a given interval. If growth is not detected after 12, 24, 48, 60, 72 or more hours in the calorimeter, the concentrated fraction can be removed from the calorimeter and placed in the secondary growth detection system (e.g., the same system that is used to interrogate the depleted/dilute/supernatant fraction). Optionally, the total time that the concentrated fraction is interrogated in both the calorimeter and the secondary growth detection system can be about 4, 5, 6 or more days. The secondary growth determination systems, which can be used for both supernatant and post-calorimetry pellet samples, can utilize any number of detection modes, which are described herein. These modes include, without limitation, optical absorbance, optical spectroscopy, optical microscopy, pH measurement, gaseous measurement, mass measurement, or electrical measurement. In some cases, both the concentrated fraction/pellet and the depleted fraction/supernatant are interrogated in parallel, and the result of the growth measurement in one or both of the fractions can be used as the basis for a determination of a microbe-positive sample. After the sample is called positive, other draws from the same patient, or any reserved portion of the original sample can be used for , e.g., microbial identification and/or antimicrobial susceptibility testing.

In embodiments according to multiple aspects of the disclosure, fresh nutrient media and/or agar may be introduced into the sample prior to the completion of the concentration step, and/or prior to monitoring either fraction or sample for microbial growth.

In yet another aspect, this disclosure relates to methods of culturing blood samples suspected of comprising microorganisms, which methods include contacting a blood sample with a resin or other material capable of adsorbing antimicrobial agents, introducing a portion of the blood sample into an isothermal calorimeter, and measuring heat flow from the sample to determine positive microorganism growth based on an absolute or relative signal. If no growth is measured (e.g., during a measurement interval), the sample is removed from the calorimeter and transferred to a secondary growth determining system as described above in connection with the preceding aspects of the disclosure. In embodiments according to this aspect of the disclosure, a liquid volume of 12, 15, 20, 25, 30, 35, 40, 45, or 50 ml may be input into the calorimeter, and/or the blood sample may comprise a volume of about 8 ml when collected in a dedicated vessel or receptable. In certain embodiments of the method, as is described in connection with the foregoing aspects, the blood sample may be contacted with nutrient media, anticoagulant and/or lytic agent; the calorimeter may be held stable at 31-40° C.; the sample may be removed from the calorimeter after no growth is measured after a pre-determined interval, the secondary growth determining systems comprise optical, pH, gaseous, or impedance methods; at least one sample drawn from the same patient is measured for microorganism growth in parallel with direct pH, gaseous, or optical methods; the lytic agent is saponin; etc, In various instances, the pre-incubation components, which include they lytic agent, the antimicrobial adsorptive material, nutrient media and the anticoagulant are mixed together before the blood sample is added, though in some cases it may be desirable to keep one or more components separate from the others prior until the blood sample is collected. Samples can be loaded into, or removed from, the calorimeter on an individual or on demand basis, or they may be added and/or removed in batches, either when samples have tested positive or when they have not tested positive after a given interval. If growth is not detected after 12, 24, 48, 60, 72 or more hours in the calorimeter, the concentrated fraction can be removed from the calorimeter and placed in the secondary growth detection system (e.g., the same system that is used to interrogate the depleted/dilute/supernatant fraction). Optionally, the total time that the concentrated fraction is interrogated in both the calorimeter and the secondary growth detection system can be about 4, 5, 6 or more days. The secondary growth determination systems, which can be used for both supernatant and post-calorimetry pellet samples, can utilize any number of detection modes, which are described herein. These modes include, without limitation, optical absorbance, optical spectroscopy, optical microscopy, pH measurement, gaseous measurement, mass measurement, or electrical measurement. In some cases, both the concentrated fraction/pellet and the depleted fraction/supernatant are interrogated in parallel, and the result of the growth measurement in one or both of the fractions can be used as the basis for a determination of a microbe-positive sample. After the sample is called positive, other draws from the same patient, or any reserved portion of the original sample can be used for, e.g., microbial identification and/or antimicrobial susceptibility testing.

In yet another aspect, the disclosure relates to methods of interrogating blood samples for microorganisms comprising separating each sample into first and second subsamples comprising first and second concentrations of microorganisms, respectively, then monitoring the first and second subsamples for microbial growth over different time intervals. In some cases, the first subsample is monitored for five days while the second subsample is monitored for fewer than five days. The first subsample may comprise a dilute or depleted fraction or a supernatant, while the second subsample may comprise a concentrated fraction or pellet. Specific embodiments according to this embodiment are substantially as described above: In certain embodiments, the first fraction is monitored using an optical growth measurement, though in other embodiments the monitoring is done by calorimetry (e.g., isothermal or differential scanning calorimetry or any other suitable calorimetry mode). The blood sample can comprise at least 8 ml in volume and is optionally collected in a dedicated receptacle (e.g., a blood culture bottle). In some cases, the blood sample is contacted with a nutrient or nutrient media, an anticoagulant and/or a lytic agent (e.g., saponin) prior to the concentration step. The first fraction may have any suitable volume, e.g., <20, <10, <8, <6, <4, <2, <1, or <0.5 ml. While the first fraction (or the backup sample) is measured for microorganism growth, it can be held at a stable temperature, e.g., between 31° and 40° Centigrade. Similarly, the backup sample can be monitored for microbial growth using, e.g., by optical (scattering or absorbance), pH, gaseous, or impedance methods. In various instances, the pre-incubation components, which include they lytic agent, the antimicrobial adsorptive material, nutrient media and the anticoagulant are mixed together before the blood sample is added, though in some cases it may be desirable to keep one or more components separate from the others prior until the blood sample is collected. As is discussed above in connection with other aspects of this disclosure, the vessel into which the blood sample is collected may be under negative pressure, and/or may comprise a gas mixture optimized for aerobic microorganism growth. Where nutrient media is used, the nutrient media can be any suitable media including without limitation tryptic soy broth. In some embodiments, the blood sample is incubated under conditions promoting microorganism growth prior to the concentration step; for clarity, in these instances, the incubation conditions are distinct from the conditions of the lysis and concentration steps. This incubation can be performed, in some cases, in a portable sample incubation system. Also as discussed above in connection with other aspects of this disclosure, the blood sample can be incubated under conditions promoting microorganism growth prior to the concentration step; for clarity, in these instances, the incubation conditions are distinct from the conditions of the lysis and concentration steps. The concentration step can comprise any suitable concentration method, including without limitation by centrifugation, filtration, flocculation, and/or magnetic separation), the concentrated fraction is again incubated under conditions that promote microbial growth. Where separation of concentrated and depleted or dilute fractions is achieved by centrifugation, it is performed at relative centripetal force (RCF) values of 1,000×g-50,000×g or 1,000×g-10,000×g. The resulting concentrated fraction/pellet can be introduced into the calorimeter at a volume of ≤20, ≤10, ≤8, ≤6, ≤4, ≤2, ≤1, or ≤0.5 ml. In some cases, fresh nutrient media and/or agar are introduced into the concentrated fraction prior to introduction into the calorimeter. calorimetry is generally, but not necessarily, performed in a dedicated vessel or receptacle rather than the vessel or receptacle used for collection and/or processing of the blood sample. Any suitable calorimetry mode may be used to detect microbial growth, including without limitation differential scanning calorimetery and isothermal calorimetry, and microbial growth can be assessed by measuring relative and/or absolute heat flows from the sample. In some cases, the choice of calorimetric mode and/or measurement of relative vs. absolute heat flows is made based on a characteristic of the patient sample or the concentrated fraction. For instance, if a sample has been subjected to pre-incubation after collection but before processing for an interval of, e.g., 3, 4, 5, 6, 7, 8, 9, or 10 or more hours, heat flow may be determined in a different manner than for samples below this threshold. Samples can be loaded into, or removed from, the calorimeter on an individual or on demand basis, or they may be added and/or removed in batches, either when samples have tested positive or when they have not tested positive after a given interval. If growth is not detected after 12, 24, 48, 60, 72 or more hours in the calorimeter, the concentrated fraction can be removed from the calorimeter and placed in the secondary growth detection system (e.g., the same system that is used to interrogate the depleted/dilute/supernatant fraction). Optionally, the total time that the concentrated fraction is interrogated in both the calorimeter and the secondary growth detection system can be about 4, 5, 6 or more days. The secondary growth determination systems, which can be used for both supernatant and post-calorimetry pellet samples, can utilize any number of detection modes, which are described herein. These modes include, without limitation, optical absorbance, optical spectroscopy, optical microscopy, pH measurement, gaseous measurement, mass measurement, or electrical measurement. In some cases, both the concentrated fraction/pellet and the depleted fraction/supernatant are interrogated in parallel, and the result of the growth measurement in one or both of the fractions can be used as the basis for a determination of a microbe-positive sample. After the sample is called positive, other draws from the same patient, or any reserved portion of the original sample can be used for, e.g., microbial identification and/or antimicrobial susceptibility testing.

In another aspect, this disclosure relates to methods of interrogating blood samples for microorganisms, comprising separating the samples into first and second subsamples comprising first and second concentrations of microorganisms, and monitoring the first and second subsamples for microbial growth over different time intervals. Embodiments according this aspect are substantially similar to the methods described above: In certain embodiments, the first fraction is monitored using an optical growth measurement, though in other embodiments the monitoring is done by calorimetry (e.g., isothermal or differential scanning calorimetry or any other suitable calorimetry mode). The blood sample can comprise at least 8 ml in volume and is optionally collected in a dedicated receptacle (e.g., a blood culture bottle). In some cases, the blood sample is contacted with a nutrient or nutrient media, an anticoagulant and/or a lytic agent (e.g., saponin) prior to the concentration step. The first fraction may have any suitable volume, e.g., ≤20, ≤10, ≤8, ≤6, ≤4, ≤2, ≤1, or ≤0.5 ml. While the first fraction (or the backup sample) is measured for microorganism growth, it can be held at a stable temperature, e.g., between 31° and 40° Centigrade. Similarly, the backup sample can be monitored for microbial growth using, e.g., by optical (scattering or absorbance), pH, gaseous, or impedance methods. In various instances, the pre-incubation components, which include they lytic agent, the antimicrobial adsorptive material, nutrient media and the anticoagulant are mixed together before the blood sample is added, though in some cases it may be desirable to keep one or more components separate from the others prior until the blood sample is collected. As is discussed above in connection with other aspects of this disclosure, the vessel into which the blood sample is collected may be under negative pressure, and/or may comprise a gas mixture optimized for aerobic microorganism growth. Where nutrient media is used, the nutrient media can be any suitable media including without limitation tryptic soy broth. In some embodiments, the blood sample is incubated under conditions promoting microorganism growth prior to the concentration step; for clarity, in these instances, the incubation conditions are distinct from the conditions of the lysis and concentration steps. This incubation can be performed, in some cases, in a portable sample incubation system. Also as discussed above in connection with other aspects of this disclosure, the blood sample can be incubated under conditions promoting microorganism growth prior to the concentration step; for clarity, in these instances, the incubation conditions are distinct from the conditions of the lysis and concentration steps. The concentration step can comprise any suitable concentration method, including without limitation by centrifugation, filtration, flocculation, and/or magnetic separation), the concentrated fraction is again incubated under conditions that promote microbial growth. Where separation of concentrated and depleted or dilute fractions is achieved by centrifugation, it is performed at relative centripetal force (RCF) values of 1,000×g-50,000×g or 1,000×g-10,000×g. The resulting concentrated fraction/pellet can be introduced into the calorimeter at a volume of ≤20, ≤10, ≤8, ≤6, ≤4, ≤2, ≤1, or ≤0.5 ml. In some cases, fresh nutrient media and/or agar are introduced into the concentrated fraction prior to introduction into the calorimeter. calorimetry is generally, but not necessarily, performed in a dedicated vessel or receptacle rather than the vessel or receptacle used for collection and/or processing of the blood sample. Any suitable calorimetry mode may be used to detect microbial growth, including without limitation differential scanning calorimetery and isothermal calorimetry, and microbial growth can be assessed by measuring relative and/or absolute heat flows from the sample. In some cases, the choice of calorimetric mode and/or measurement of relative vs. absolute heat flows is made based on a characteristic of the patient sample or the concentrated fraction. For instance, if a sample has been subjected to pre-incubation after collection but before processing for an interval of, e.g., 3, 4, 5, 6, 7, 8, 9, or 10 or more hours, heat flow may be determined in a different manner than for samples below this threshold. Samples can be loaded into, or removed from, the calorimeter on an individual or on demand basis, or they may be added and/or removed in batches, either when samples have tested positive or when they have not tested positive after a given interval. If growth is not detected after 12, 24, 48, 60, 72 or more hours in the calorimeter, the concentrated fraction can be removed from the calorimeter and placed in the secondary growth detection system (e.g., the same system that is used to interrogate the depleted/dilute/supernatant fraction). Optionally, the total time that the concentrated fraction is interrogated in both the calorimeter and the secondary growth detection system can be about 4, 5, 6 or more days. The secondary growth determination systems, which can be used for both supernatant and post-calorimetry pellet samples, can utilize any number of detection modes, which are described herein. These modes include, without limitation, optical absorbance, optical spectroscopy, optical microscopy, pH measurement, gaseous measurement, mass measurement, or electrical measurement. In some cases, both the concentrated fraction/pellet and the depleted fraction/supernatant are interrogated in parallel, and the result of the growth measurement in one or both of the fractions can be used as the basis for a determination of a microbe-positive sample. After the sample is called positive, other draws from the same patient, or any reserved portion of the original sample can be used for , e.g., microbial identification and/or antimicrobial susceptibility testing.

In another aspect, this disclosure relates to an automated method for determining microorganism growth in samples of human origin comprising introducing the following components into a vessel prior to centrifugation: a sample of human origin, such as blood, cerebrospinal fluid, synovial fluid, plural fluid, pericardial fluid; one or more components capable of lysing eukaryotic cells; one or more barrier fluids, defined as a water-miscible or water-immiscible fluid or solution with a density and/or viscosity such that it partitions below lysed blood cells during centrifugation; a plurality of lysed blood cells cannot enter the layer it forms; and microorganisms may enter the layer it forms during centrifugation; optionally, a cushioning fluid, defined as a water-miscible or water-immiscible fluid or solution with a density greater than that of a plurality of eukaryotic and prokaryotic cells; optionally, one or more anticoagulants; and optionally, one or more anti-foaming components; into a collection tube comprising the sample of human origin. The method further comprises centrifuging the mixture under conditions suitable to enable microorganisms to enter the barrier fluid; removing the blood cell layer and introducing it into a nutrient media capable of supporting microorganism growth and performing one or more discrete or continuous monitoring methods for determining microorganism growth during incubation under conditions promoting microorganism growth; and introducing a plurality of the remaining microorganisms in the centrifuged vessel to a nutrient media capable of supporting microorganism growth and performing one or more discrete or continuous monitoring methods for determining microorganism growth during incubation under conditions promoting microorganism growth.

Another aspect of this disclosure relates to an automated method for determining microorganism growth in samples of human origin comprising: introducing a blood sample or treated blood sample into a vessel comprising a liquid or semi-solid filtering layer; centrifuging the mixture under conditions suitable to enable microorganisms to enter the filtering layer but that prevent a plurality of blood cells from entering the filtering layer; removing layer comprising blood cells above the filtering layer and introducing it into a nutrient media capable of supporting microorganism growth and performing one or more discrete or continuous monitoring methods for determining microorganism growth during incubation under conditions promoting microorganism growth; and introducing a plurality of the microorganisms present in the centrifuged vessel to a nutrient media capable of supporting microorganism growth and performing one or more discrete or continuous monitoring methods for determining microorganism growth during incubation under conditions promoting microorganism growth.

In some embodiments: the blood sample comprises sodium polyanethole sulfonate (SPS) and/or citrate as an anticoagulant; blood sample treatment comprises mixing with SPS and saponin; the blood sample treatment comprises mixing with polypropylene glycol; blood sample treatment comprises mixing with one or more nutrient media; blood sample treatment comprises sample incubation under conditions promoting microorganism growth; wherein a liquid filtering layer comprises one or more thickening agents.

In an embodiment, a thickening agent includes but is not limited to a simple sugar, a sugar polymer, a linear polymer, a branched polymer. In an embodiment, a thickening agent is sucrose. In an embodiment, a liquid filtering layer comprises one or more thermally responsive gelling agents. In an embodiment, a gelling agent comprises gelatin. In an embodiment, a liquid filtering layer comprises heavy water. In an embodiment, two or more liquid filtering layers are present. In an embodiment, a liquid filtering layer is non-water miscible. In an embodiment, the liquid filtering layer comprises a silicone oil, fluorinated surfactant, fluorinated solvent, or fluorinated polymer. In an embodiment, a liquid filtering layer can be pipetted as a liquid above the temperature of 10, 20, 30, 35° C. In an embodiment, the blood sample is added at a temperature below said temperatures. In an embodiment, the blood sample is added and, following centrifugation, the blood cell containing layer is removed below the temperature at which the liquid filter layer is removed. In an embodiment, centrifugation is performed in a swinging bucket rotor. In an embodiment, centrifugation is performed between 500-6,000×g. In an embodiment, the method for monitoring microorganism growth is optical, including absorbance, fluorescence, fluorescence polarization, time-resolved fluorescence, nephelometry, luminescence, forward laser scatting, multi-angle light scattering, dynamic light scattering, high-resolution imaging; mass resonance; acoustic; electronic, including impedance, voltammetry, amperometry. In an embodiment, different methods are used for monitoring microorganism growth for the two different layers. In an embodiment, one or more biochemical probes are added. In an embodiment, the probes comprise probes that have fluorescent properties that are modified by growing microorganisms, probes that can associate with microorganism surfaces, probes that can selectively penetrate live or dead microorganisms. In an embodiment, the one or more probes detect the function of one or more enzymes including but not limited to enzymes responsible for peptidoglycan assembly, disassembly, reassembly; esterases; phosphatases; galactosidase; peptidases; ureases; catalases; gelatinases; hydrolysases; DNases; lipases; proteases; oxidoreductases. In an embodiment, two probes form a FRET pair. In an embodiment, the conditions that promote microorganism growth comprise incubation at 33-37° C., agitation, and the presence or absence of oxygen.

In another aspect, the disclosure relates to a method for culturing blood samples suspected of comprising microorganisms, comprising contacting a blood sample with resins capable of adsorbing antimicrobial agents; performing at least one centrifugation step using at least one aqueous-miscible high-density layer to partition microorganisms in a higher-density layer and thereby separate them from lower-density blood cell components in a supernatant above the layer and larger, similar-density blood cell components in a pellet below the layer; measuring the microorganisms partitioned into the higher-density layer for growth during incubation; and retaining a plurality of the supernatant, the pellet, and any remainder of the high-density layer comprising microorganisms following centrifugation in a “backup” sample also monitored for microorganism growth during incubation. In an embodiment, the growth measurement is performed optically. In an embodiment, the growth measurement is performed by calorimetry. In an embodiment, the blood sample comprises at least about 8 mL in volume and is collected in a receptacle. In an embodiment, the blood sample is contacted with nutrient media and an anticoagulant. In an embodiment, the blood sample is contacted with a lytic agent capable of selectively lysing mammalian cells. In an embodiment, the high-density aqueous layer is a high-concentration sugar, salt, or polymer including, but not limited to, sucrose, cesium, Ficoll-Pacque. In an embodiment, the high-density aqueous layer comprises nutrient media or one or more microorganism growth-promoting components. In an embodiment, the high-density aqueous layer is approximately equal, half, quarter, one fifth, one eighth the volume of the volume of the blood plus media. In an embodiment, the high-density aqueous layer is not present in the receptacle into which the blood is collected from the patient. In an embodiment, the microorganisms are separated from a portion of the high-density aqueous layer before the onset of growth determining steps. In an embodiment, the pellet is held stable at 31-40° C. during measurement of microorganism growth. In an embodiment, the retained supernatant and remainder of the pellet are monitored for growth by optical, pH, gaseous, or impedance methods. In an embodiment, the optical methods comprise scattering or absorbance. In an embodiment, calorimetry comprises isothermal or differential scanning calorimetry. In an embodiment, the lytic agent is saponin. In an embodiment, the anticoagulant is one or more of sodium polyanethole sulfonate (SPS) and citrate In an embodiment, the resin is isolated from the liquid reagents in the receptacle prior to the addition of blood sample to the receptacle. In an embodiment, the antimicrobial-adsorbing resin is isolated from the SPS and/or the nutrient media prior to the addition of blood sample to the receptacle. In an embodiment, the receptacle is under negative pressure such that the blood sample fills the receptacle when connected with a standard butterfly fitting to a venous IV. In an embodiment, a gas mixture in the receptacle is optimized for aerobic microorganism growth. In an embodiment, the nutrient media is a tryptic soy broth. In an embodiment, the resin is magnetic. In an embodiment, the resin is supported on a solid substrate. In an embodiment, the blood sample is incubated under conditions promoting microorganism growth prior to the concentration step. In an embodiment, growth prior to the concentration step is performed differently than the manner in which the concentration step is performed. In an embodiment, growth prior to the concentration step is performed in a portable system. In an embodiment, the microorganisms is incubated under conditions promoting microorganism growth following completion of the centrifugation steps. In an embodiment, centrifugation is performed at a speed of ≤1,000×g, 1,000×g, 1,500×g, or 2,000×g. In an embodiment, the combination of the supernatant and blood cell pellet is measured for microorganism growth using at least one of optical absorbance, optical spectroscopy, optical microscopy, pH measurement, gaseous measurement, mass measurement, or electrical measurement. In an embodiment, supernatant/pellet growth measurement is performed approximately in parallel with the high-density aqueous layer growth measurement. In an embodiment, the result of the growth measurement from one or more of the concentrate and supernatant samples is returned to the user. In an embodiment, microorganism identification and/or antimicrobial susceptibility testing and/or antimicrobial resistance determinations are performed following the determination of positive growth. In an embodiment, the method is automated.

Another aspect of this disclosure relates to A method for preparing two or more samples for microbial growth determination from a patient-derived blood sample, comprising: passing a fluid comprising whole blood from the blood sample through one or more aqueous-miscible high-density layers, such that a portion of a first blood component within the sample passes through one or more of the aqueous-miscible high-density layers, a portion of a second blood component does not enter one or more of the aqueous-miscible high-density layers, and a portion of the microorganism from the sample is retained by one or more of said aqueous-miscible high-density layers; and collecting at least a portion of the aqueous-miscible high-density layers comprising the microbe as the first sample for microbial growth monitoring; collecting the remainder of the sample as the second sample for microbial growth monitoring. In an embodiment, In an embodiment, the portion of the density layer comprising the microbe is substantially free of blood components that interfere with scattering or absorption based measurements of microbial growth. In an embodiment, the method includes centrifugation of the sample before collection. In an embodiment, centrifugation is performed at a speed of ≤1,000×g, 1,000×g, 1,500×g, or 2,000×g. In an embodiment, centrifugation is performed between the speeds of 300 and 800×g. In an embodiment, the microbe containing portion is collected for growth measurement. In an embodiment, the growth measurement is performed optically. In an embodiment, a fluid comprising the whole blood that is not within the portion of the density layer collected is collected for growth measurement. In an embodiment, the growth measurement is performed by calorimetry.

In another aspect, this disclosure relates to a microbial inoculate prepared according to a method disclosed herein. In an embodiment, the blood sample is contacted with a resin. In an embodiment, the blood sample comprises at least about 8 mL in volume and is collected in a receptacle. In an embodiment, the blood sample is contacted with nutrient media and an anticoagulant. In an embodiment, the blood sample is contacted with a lytic agent capable of selectively lysing mammalian cells. In an embodiment, a high-density aqueous layer is a high-concentration sugar, salt, or polymer including, but not limited to, sucrose, cesium, Ficoll-Pacque. In an embodiment, two or more high-density aqueous layers are layered above one another. In an embodiment, the multiple high-density aqueous layers have increasing densities from top-to-bottom. In an embodiment, a high-density aqueous layer comprises nutrient media or one or more microorganism growth-promoting components. In an embodiment, a high-density aqueous layer is approximately equal, half, quarter, one fifth, one eighth the volume of the volume of the blood plus media. In an embodiment, the microorganisms are separated from a portion of the high-density aqueous layer before the onset of growth determining steps. In an embodiment, the optical methods comprise scattering or absorbance. In an embodiment, calorimetry comprises isothermal or differential scanning calorimetry. In an embodiment, the lytic agent is saponin. In an embodiment, the anticoagulant is one or more of sodium polyanethole sulfonate (SPS) and citrate. In an embodiment, the nutrient media is a tryptic soy broth. In an embodiment, the resin is magnetic. In an embodiment, the resin is supported on a solid substrate. In an embodiment, the blood sample is incubated under conditions promoting microorganism growth prior to the step of passing a fluid comprising whole blood from the blood sample through an aqueous-miscible high-density layer. In an embodiment, the microorganisms is incubated under conditions promoting microorganism growth following completion of the centrifugation steps. In an embodiment, supernatant/pellet growth measurement is performed approximately in parallel with the high-density aqueous layer growth measurement. In an embodiment, microorganism identification and/or antimicrobial susceptibility testing and/or antimicrobial resistance determinations are performed following the determination of positive growth. In an embodiment, the method is automated.

In yet another aspect, this disclosure relates to a method for preparing a microbial inoculate from a blood sample, comprising: passing a fluid comprising whole blood from the blood sample through an aqueous-miscible high-density layer, such that a first blood component within the sample passes through the aqueous-miscible high-density layer, a second blood component does not enter the aqueous-miscible high-density layer, and a microorganism from the sample is retained by said aqueous-miscible high-density layer; and collecting at least a portion of the aqueous-miscible high-density layer comprising the microbe. In an embodiment, the portion of the density layer comprising the microbe is substantially free of blood components that interfere with scattering or absorption based measurements of microbial growth. In an embodiment, the step of passing the fluid comprising whole blood from the blood sample through the aqueous-miscible high-density layer comprises centrifugation of the sample. In an embodiment, centrifugation is performed at a speed of ≤1,000×g, 1,000×g, 1,500×g, or 2,000×g. In an embodiment, centrifugation is performed between the speeds of 300 and 800×g. In an embodiment, the microbe containing portion is collected for growth measurement. In an embodiment, the growth measurement is performed optically. In an embodiment, a fluid comprising the whole blood that is not within the portion of the density layer collected is collected for growth measurement. In an embodiment, the growth measurement is performed by calorimetry. In an embodiment, the blood sample is contacted with a resin. In an embodiment, the blood sample comprises at least about 8 mL in volume and is collected in a receptacle. In an embodiment, the blood sample is contacted with nutrient media and an anticoagulant. In an embodiment, the blood sample is contacted with a lytic agent capable of selectively lysing mammalian cells. In an embodiment, a high-density aqueous layer is a high-concentration sugar, salt, or polymer including, but not limited to, sucrose, cesium, Ficoll-Pacque. In an embodiment, two or more high-density aqueous layers are layered above one another. In an embodiment, the multiple high-density aqueous layers have increasing densities from top-to-bottom. In an embodiment, a high-density aqueous layer comprises nutrient media or one or more microorganism growth-promoting components. In an embodiment, a high-density aqueous layer is approximately equal, half, quarter, one fifth, one eighth the volume of the volume of the blood plus media. In an embodiment, the microorganisms are separated from a portion of the high-density aqueous layer before the onset of growth determining steps. In an embodiment, the optical methods comprise scattering or absorbance. In an embodiment, calorimetry comprises isothermal or differential scanning calorimetry. In an embodiment, the lytic agent is saponin. In an embodiment, the anticoagulant is one or more of sodium polyanethole sulfonate (SPS), citrate. In an embodiment, the nutrient media is a tryptic soy broth. In an embodiment, the resin is magnetic. In an embodiment, the resin is supported on a solid substrate. In an embodiment, the blood sample is incubated under conditions promoting microorganism growth prior to the step of passing a fluid comprising whole blood from the blood sample through an aqueous-miscible high-density layer. In an embodiment, the microorganisms is incubated under conditions promoting microorganism growth following completion of the centrifugation steps. In an embodiment, supernatant/pellet growth measurement is performed approximately in parallel with the high-density aqueous layer growth measurement. In an embodiment, microorganism identification and/or antimicrobial susceptibility testing and/or antimicrobial resistance determinations are performed following the determination of positive growth. In an embodiment, the method is automated.

In an aspect, the disclosure relates to a microbial inoculate prepared according to the foregoing method.

The foregoing listing is intended to be exemplary rather than exhaustive, and those of skill in the art will appreciate that other aspects and embodiments not described above are within the scope of the invention.

### BRIEF DESCRIPTION OF THE DRAWINGS

The drawings presented in this disclosure are intended to be exemplary, rather than limiting. Unless otherwise indicated, drawings are not necessarily to scale.

FIG. 1 shows sample tubes comprising 40%, 50% or 60% sucrose density layers, illustrating the partitioning of microbes and red blood cells or hemoglobin.

FIG. 2 shows the supernatant, interface, and filter layers of a sample tube following centrifugation according to an embodiment of this disclosure.

FIG. 3 illustrates the percentage of microorganisms in the layers of centrifuged sample tubes comprising 40%, 50% or 60% sucrose density layers.

FIG. 4 shows of centrifuged sample tubes inoculated with the indicated bacterial species and illustrates the partitioning of the bacterial species and blood components.

FIG. 5 shows centrifuged sample tubes inoculated with microbes, whole blood or lysed blood after 1 hour, 2 hours and 2.5 hours of centrifugation time.

FIG. 6 shows centrifuged sample tubes according to an embodiment of this disclosure.

FIG. 7 shows heat flows measured from microbial inoculate samples from whole blood (P2), processed blood (P1) and TSB as described in Example 8.

FIG. 8 shows heat flows from negative controls processed as in Example 8.

FIG. 9 is a schematic illustration of samples prepared as described in Example 9.

FIG. 10 shows absorbance (OD600)-based growth determination of three species of bacteria spiked into blood and processed following the lysis-centrifugation-liquid culture method disclosed here.

FIG. 11 shows absorbance (OD600)-based growth determination of three species of bacteria spiked into blood and processed following the lysis-centrifugation-liquid culture method disclosed here and further shows the time-to-positivity of a BACTEC® 9050 for the same three spiked samples.

FIG. 12 shows the computer aided design of a system capable of performing the methods described here.

FIG. 13 shows fluorescence-based growth determination of three species of bacteria spiked into blood and processed following the lysis-centrifugation-liquid culture method disclosed here based on their abilities to reduce resazurin.

FIG. 14A depicts, schematically, probabilities of capturing at least one bacterium in a concentrated fraction of a blood sample against the number of bacteria in the sample across three different rates of capture. FIG. 14B shows the probabilities of finding a given number of bacteria in a concentrated fraction when bacteria partition into that fraction at rates of 75% and 50%, respectively.

### DETAILED DESCRIPTION

### Overview

This disclosure relates, generally, to rapid detection of microorganism growth in blood samples and blood-derived samples. Certain methods according to this disclosure are based on two concepts, which may be combined or used independently. The first concept relates to dividing an incoming blood sample or blood-derived sample into two components, and monitoring each of these components for growth. Without wishing to be bound by any theory, the inventors have found that a single patient blood sample, approximately 8-10 cc of blood, can be divided into two or more subsamples, each of which may be cultured in similar media, in such a way that a first subsample may detect growth faster than the other(s) while a second subsample may be more sensitive at detecting growth where very low numbers of bacteria are present than the other(s). It should be noted that lower blood volumes derived from some patients, such as pediatric patients, may be treated similarly.

The second concept relates to the use of isothermal calorimetry to determine microorganism growth. According to certain embodiments of this disclosure, isothermal calorimetry may be used to determine microorganism growth following one or more processing steps that, e.g., concentrate microorganisms, such that only a portion of the original sample is measured by calorimetry for microorganism growth. The remainder of the sample may be retained for monitoring using one or more different microorganism growth detection modalities, such as optical methods.

The third concept relates to the collection of patient blood samples and the conditions under which those samples are held between the time of their collection and the time they are processed and interrogated for growth. Standard clinical practice today is to hold collected samples at room or ambient temperature until they arrive in a clinical laboratory, where they are processed and monitoring for microbial growth begins. In current practice in U.S. hospitals, this is achieved by loading the samples into a blood culture machine that monitors growth while incubating samples. As is discussed in greater detail below, in some embodiments of this disclosure patient blood samples may be held in conditions supporting microbial growth, e.g., incubated above ambient temperature, with nutrients or nutrient media added, with materials (e.g., resins) that adsorb or inactivate antimicrobial agents that may be present in the patient's blood, etc. These approaches are referred to generally as “pre-incubation” or “pre-treatment.”

Methods that concentrate blood samples prior to determining microorganism growth are described in U.S. Pat. No. 5,070,014 and known as the “lysis-centrifugation” technique. While this approach offered fast times to detection (TTDs) because it concentrated microorganisms, selectively lysed mammalian cells, and removed substances toxic to microorganism growth, it suffered from lower positive predictive values (PPVs) than continuous monitoring systems [Pohlman et al. J. Clin. Microbiol. 33 (1995): 2525-2529].

In order to gain the benefit of speed conferred by the lysis-centrifugation technique without compromising PPVs, the inventors have found it useful to retain not only the pellet resulting from a centrifugation step, but also the supernatant. Without wishing to be bound by any theory, it is believed that this design helps ensure that more of the microorganisms present in the initial blood sample are retained, while simultaneously enabling a concentrated portion of the initial sample to be evaluated for rapid microorganism detection.

The division of a patient blood sample or blood-derived sample may also be advantageous for performing calorimetry, which may be performed directly on the collected blood sample or with one or more additives, including: nutrient media, anticoagulant, antimicrobial adsorbing resin, lytic components. Concentrating microorganisms from the sample prior to their introduction into the calorimeter may be advantageous for speeding time to microorganism growth detection and easing engineering constraints insofar as concentration may facilitate (a) maximization of the number of microbes interrogated and (b) the concentration of those microbes in a reduced volume. Reduced volumes may also advantageously decrease the time required for temperature equilibration, an important precondition for accurate isothermal calorimetry, as well as easing the engineering constraints on the calorimeter design, since smaller volumes are more easily controlled. Additionally, when microorganism growth is detected, a smaller volume may be advantageous for downstream processing.

The inventors have also found anticoagulant treatment and/or selective lysis of mammalian cells to be uniquely advantageous in calorimetric detection of microbial growth. Just as all bacteria and yeast produce heat, so do blood cells and platelets: erythrocytes at 0.01 pW/cell, platelets at 0.06 pW/cell, and leukocytes at 5 pW/cell [Biocalorimetry: Foundations and Contemporary Approaches. Bastos, M., Ed. United States: CRC Press, 2016]. Without wishing to be bound by any theory, it is believed that mammalian cells in a blood or blood-derived sample may be selectively inhibited through the use of anticoagulants such as sodium polyanethol sulfonate (SPS) and/or mammalian-selective lysing agents such as saponin. As is discussed in greater detail below, blood samples prepared by lysis-centrifugation methods described herein exhibit negative heat flows when examined calorimetrically. Without wishing to be bound by any theory, it is believed that the lysis of mammalian cells, including red blood cells (RBCs) results in micellization of mammalian cell membranes, an endothermic process at some elevated temperatures [Biocalorimetry: Foundations and Contemporary Approaches. Bastos, M., Ed. United States: CRC Press, 2016]. Against the backdrop of such negative heat flows, microbial growth is readily called as any positive trend in heat flow.

As is discussed in greater detail below, the current standard for positive blood culture assessment is continuous monitoring of metabolic indicators of microbial growth such as pH or CO₂. Fluorescence quenching, for example, is implemented in the Bactec™ series of blood culture instruments and consumables (Becton-Dickinson, Sparks Md.). Continuous monitoring systems rely on time series measurements to detect changes over time that indicate increased microbial metabolic activity. These approaches, by their nature, require samples to be measured over intervals sufficient to permit one or more bacterial doublings. By contrast, the calorimetric determination methods described herein may permit very rapid determinations of the presence of microbes in a sample.

Viewed in the context of their potential for rapid culture determinations, the calorimetry-based methods of this disclosure are uniquely complemented by the pre-incubation methods described herein. The current clinical practice of holding collected blood samples at room temperature prior to laboratory processing and measurement is predicated, in part, on the desirability of achieving log-phase growth during continuous monitoring processes, in order to differentiate the microbial signal from the mammalian cell baseline. Holding samples at reduced temperatures lowers the rate of microbial growth, thus reducing the risk that microbes will reach stationary phase before they enter the blood culture system. By contrast, in the “rapid-read” calorimetry methods described herein, it may be valuable to maximize the potential thermal signal of microbes by maximizing the number of microbes present in the sample when it is placed in the calorimeter. Thus, pre-incubation under conditions that favor microbial growth may increase both the magnitude and rate of growth of positive heat flows by the time the sample is placed in the calorimeter. This, in turn, may reduce the time required to achieve a positive result and/or reduce the risk of false negative results.

In some embodiments according to this disclosure, antimicrobial-adsorbing resins are included with the lysis-centrifugation technique.

Another aspect of this disclosure relates to the observation that different blood samples drawn from the same patient at approximately the same time may not only be incubated under different media and gas conditions, as is the current standard, but may also be processed and/or assessed for microorganism growth with different methods. For example, aerobic samples may be processed by lysis-centrifugation followed by calorimetry while anaerobic samples may be processed by lysis-centrifugation alone or without centrifugal concentration.

### Lysis-Centrifugation and Preparation of Samples or Subsamples

As is known to those skilled in the art, when the lysis-centrifugation approach is performed on a whole blood sample with sodium polyanethole sulfonate (SPS) as the anticoagulant and saponin as the lysing agent, a pellet of approximate volume 1.5-2 mL consisting primarily of “saponin ghosts,” as termed in the literature, is obtained. The effective viscosity of this layer decreases the probability that a single bacteria (if, for example, there were only to be a single bacteria in the 10 cc of drawn blood) would sediment through the entire layer and reach the tube bottom. Thus, it becomes necessary to remove at least a portion of this layer when performing blood culture.

In U.S. Pat. No. 3,928,139, Dorn described an alternative to the lysis-centrifugation procedure as it is currently known, wherein microbes were collected in a higher-density fluid that partitioned below the saponin ghost pellet during centrifugation. This fluid effectively served as a “liquid filter” that retained bacteria but prevented a plurality of blood cells from entering. The examples in U.S. Pat. No. 3,928,139 show moderate bacterial retention and no results for yeast or fungi and the approach was not commercialized.

Embodiments of the present disclosure are based on the inventors' discovery that separate cultures may be prepared from each layer of a multi-layer partition achieved by centrifugation either preceding or following blood lysis. The partitioning fluid may be a liquid or semi-solid gelatinous layer that has higher viscosity and/or density than water. In an embodiment, this layer is a thermally sensitive gel comprising 40-60% sucrose with 0.5-5% gelatin. The thermally responsive gel may be designed to be gelatinous at room temperature and flow substantially more easily above a higher temperature that is compatible with microorganisms, around 35-40° C. Without wishing to be bound by any theory, this design may be advantageous for creating a cleaner separation between layers during removal of the layer above the partitioning fluid following centrifugation.

Centrifugation of a bacteria-comprising blood sample in a tube comprising the above-mentioned partitioning layer at 500-5,000×g for 5-120 minutes will result in a partitioning of a percentage of microorganisms into the partitioning layer. This layer, however, is designed to prevent the entry of blood cells. Thus, in this way, following centrifugation the partitioning layer comprises microorganisms and is free of a majority of blood cells. This layer is termed the “red blood cell (RBC)-depleted” layer. Provided the sample comprises sufficient numbers of microorganisms to penetrate the saponin ghost layer during centrifugation, this bottom layer may be microorganisms-rich and be separated from those cells.

The layer comprising the plurality of RBCs will reside above the RBC-depleted layer following centrifugation. For samples comprising very few microorganisms, and most specifically in the case of yeast or fungi, it is likely these cells may reside in this “RBC layer.”

As is known to those skilled in the art, an additional plasma layer will reside above the RBC layer following centrifugation. This layer should not comprise microorganisms and will be discarded following centrifugation.

The lytic reagents may be added before or after centrifugation. As known to those skilled in the art, these may comprise saponin, SPS, and polypropylene glycol (PPG), added as an anti-foaming agent.

Additionally, a cushioning fluid, as known to those skilled in the art, may also be included to maximize microbial retention in subsequent culture. During centrifugation, the silicone oil mixture will partition below a plurality of the saponin ghosts and bacteria will partition in and below the silicon oil.

The inventors have observed differences between the layers in terms of microorganism retention as well as RBC retention, and have discovered that fast times to positivity can be achieved without compromising the number of positive samples by culturing both the RBC-depleted and the RBC layers separately. Because it contains comparatively few RBCs, the RBC-depleted layer may facilitate rapid indication of microbial growth. For example, if optical methods are used for detection of growth in liquid culture, positive microbial growth may be identified at counts of approximately 10²-10⁵ CFU/mL. At the same time, however, measuring growth from the RBC-depleted layer alone may result in false negatives for those samples not comprising sufficient microbes (or microbes of sufficient density) to partition in the RBC-depleted layer. Embodiments of this disclosure address this risk by interrogating the RBC-rich layer for microbial growth in parallel with the RBC-depleted layer. Without wishing to be bound by any theory, in those instances where a sample comprises very few microbes, or where microbes in the sample do not partition into the RBC-depleted layer efficiently, the RBC-rich layer may comprise a significant fraction of the total microbes in the sample. Interrogation of the RBC-rich layer should therefore allow a higher sensitivity with respect to detecting positive growth for samples comprising few microbes. However, because of the large numbers of RBCs in these samples, the limits of detection may be reduced to, for example, approximately 10⁶-10⁸ CFU/mL.

A variety of suitable methods may be employed for partitioning the layers. In addition to sucrose, non-aqueous miscible solvents, such as silicone oils or fluorocarbons, aqueous solutions or solvents may be used. These may include, but are not limited to, deuterated water or solutions of >10% sugars, Ficoll, dextran, Percoll, hypaque sodium (3,5-diacetamido-2,4,6-triiodobenzoic acid sodium salt).

Cushioning fluids may comprise water miscible or immiscible liquids or solutions with densities >1.08 or >1.1. These may include, but are not limited to, fluorocarbons, deuterated water, and silicone oils.

While the foregoing disclosure of sample preparation has focused specifically on blood culture, the methods and systems described herein may be applied to a variety of sample types and to samples of any origin, including without limitation cerebrospinal fluid and other sterile body fluids.

### Sample Preparation and Adsorptive Materials

In certain embodiments of the present disclosure, whole blood is collected into a consumable comprising an anticoagulant, such as sodium polyanethole sulfonate (SPS); antimicrobial-adsorbing resins; nutrient media; and a lytic agent, such as saponin. Additionally, one or more anti-foaming materials, such as polypropylene glycol, may be included. The consumable may be designed to specifically draw the blood sample in and may further have a pre-determined gas ratio present, as known to those skilled in the art to enhance microorganism growth. The saponin may be present at any concentration ranging from 0.30 to 0.26%w/v.

Certain sample preparation methods of the present disclosure utilize resins designed to adsorb antimicrobial compounds, such as those disclosed in U.S. Pat. No. 4,174,277 and U.S. Pat. No. 5,624,814 and fully incorporated by reference herein. While such resins are standard components of continuous monitoring blood culture consumable formulations, as known to those skilled in the art they readily adsorb lytic agents, such as saponin, during storage. Thus, in order to maximize the antimicrobial adsorption potential of the resins, they may be isolated from saponin and one or more other of the other consumable constituents components prior to blood addition into the consumable. Additionally, in order to maximize compatibility with centrifugation, resins may be supported on solid substrates or may be capable of magnetic capture.

In some embodiments a non-aqueous-miscible cushioning fluid with a density greater than that of water may be used. These are described in U.S. Pat. No. 4,212,948 and incorporated fully herein by reference. Aqueous fluids with tuned viscosities may also be employed, as described in U.S. Pat. No. 3,928,139 and incorporated fully herein by reference. Such fluids may be advantageous for maximizing microorganism yield during centrifugation and/or during sample separation.

In some embodiments an aqueous-miscible fluid with a density greater than that of water, for example sucrose, cesium salt, or Ficoll-Pacque dissolved in water, may be used to separate microorganisms from blood cells by using a timed centrifugation. A physical barrier or frit may be used to keep the dense layer separated while allowing entry during centrifugation. As is known to those skilled in the art, by layering a blood sample comprising microorganisms above such a higher-density layer, a timed centrifugation may be used to separate bacteria, with densities greater than that of white blood cells and platelets but approximately equal to that of red blood cells, from all these species. Specifically, by selecting a centrifugation speed sufficiently low to prevent white blood cells and platelets from penetrating the dense liquid layer and by taking advantage of the fact that smaller bacteria will travel more slowly through the layer than larger red blood cells, by creating a sufficiently long dense layer path length and halting the centrifugation before the bacteria have collected at the red blood cell pellet at the bottom of the layer, a portion of the bacteria devoid of most mammalian cells will be retained in the dense layer. This process may be performed with non-lysed blood or may be performed with lysed blood, in which the density of a portion of the red blood cells is decreased below that of bacteria so the centrifugation speed may be changed.

In some embodiments the aqueous-miscible, higher-density fluids may comprise one or more nutrient media or components of nutrient media to promote microorganism growth.

In an embodiment, following the introduction of blood into the consumable, incubation under conditions promoting microorganism growth may be performed. Alternatively, a mixing step may be performed prior to the onset of and/or during incubation. This step may be performed by any known method including, but not limited to, mechanical shaking, such as with an orbital or rotary shaker, mechanical rolling, or magnetic stirring. In one embodiment, the resins may be magnetic. Conditions promoting microorganism growth may include temperatures of 31-39° C. and/or mixing. The incubation and/or mixing steps may be performed in a dedicated incubation device. In alternative embodiments, samples need not be incubated prior to centrifugation.

In an embodiment samples are then subjected to a concentration step. Any concentration method including, but not limited to, centrifugation, filtration, flocculation, magnetic separation, may be used. A serum separator tube may be used. Centrifugation may include differential centrifugation. In an embodiment, centrifugation at >1000×g or 1000-10,000×g, is performed. Following centrifugation the concentrate is separated from the supernatant. For the purposes of this method where a specific pellet may not be formed, the “concentrate” is defined a set volume remaining after supernatant separation. The concentrate volume may be set to be less than or equal to that of the calorimeter cell. In particular, this may be ≤6 mL, ≤4 mL, ≤2 mL, ≤1 mL, ≤0.5 mL. For the purposes of this method the “supernatant” is defined as all sample material not present in the concentrate. A filter or selective aspiration may be used to retain resins in the supernatant. Alternatively, a magnet may be used to retain magnetic resins. Alternatively, the resins may be present in the concentrate.

### Separation of Subsample Workflows

Embodiments of this disclosure relate to methods of interrogating blood samples for microorganisms. These methods may comprise, generally, separating the sample into first and second subsamples comprising first and second concentrations of microorganisms, and monitoring the first and second subsamples for microbial growth over different time intervals and/or using detection means of different sensitivity.

In some embodiments, the first subsample may be monitored for up to five days and the second subsample is monitored for fewer than five days. The sample may be separated into a pellet and a supernatant. At least one subsample may be monitored using isothermal calorimetry. If no growth is detected after the time interval, the subsample may be monitored by another monitoring method and, optionally, said subsample may be monitored until the subsample has been monitored for a total of five days. The method may further comprise contacting at least one of the sample, the first subsample, or the second subsample with a resin capable of adsorbing antimicrobial agents. The sample may comprise at least 8 mL.

As discussed above, subsample preparation may involve one or more of the following steps: contacting a blood sample with a resin capable of adsorbing antimicrobial agents; performing one or more concentration steps to concentrate microorganisms into: a) a pellet, and b) a residual supernatant; introducing a subsample comprising or derived from at least a portion of the pellet into an isothermal calorimeter; measuring heat flow from the subsample; measuring positive microorganism growth based on an absolute or relative signal; and retaining a portion of the supernatant following the concentration step in a “backup” or second subsample that is also monitored for growth, optionally using a different (e.g., less sensitive) method than isothermal calorimetry and/or monitoring the second subsample for a different interval of time than the first subsample.

Turning first to the blood sample, as discussed elsewhere in this specification, a blood sample may comprise at least about 8 mL in volume and is collected in a receptacle. The blood sample may be contacted with nutrient media, an anticoagulant, and a lytic agent capable of selectively lysing mammalian cells.

The first subsample may incorporate all or part of pellet, and the volume of the first subsample may be about 0.5, 0.7, 1, 2, 3, 4, 5, 6, 7, 8, 9, 10 mL. These comparatively low sample volumes are well suited for use in the isothermal calorimeter, which can be held stable at 31-40° C. In some embodiments, if no growth is measured in the calorimeter (e.g., during a fixed interval of 0.5, 1, 2, 3, 4, 5, 6 or more days), the sample may be removed from the calorimeter and transferred to one or more secondary growth determining systems.

Secondary growth determining systems may comprise optical, pH, gaseous, or impedance methods, and will generally (though not necessarily) incorporate a technology other than calorimetry.

The retained supernatant and remainder of the pellet may be monitored for growth by any suitable means, including optical, pH, gaseous, or impedance methods. However, it may be desirable to utilize a growth detection technology that is less sensitive, less costly per sample, and/or less resource constrained than the method used to interrogate the first subsample comprising the pellet.

At least one sample drawn from the same patient may be measured for microorganism growth in parallel with direct pH, gaseous, or optical methods. As discussed above, this sample may be prepared in any suitable manner, including without limitation using a lytic agent (e.g., saponin); an anticoagulant such as sodium polyanethole sulfonate (SPS) and/or citrate.

When utilized, adsorptive resins raise special considerations for sample preparation. Among these is the need to balance efficient removal of any systemically-administered antimicrobials in the patent blood sample (e.g., in order to avoid loss of microbes in the sample) with the need for removal of all or substantially all of the resin from the sample or subsample (e.g., to avoid confounding results in downstream phenotypic AST assays). Thus, in certain embodiments, the resin may be isolated from the liquid reagents in a receptacle prior to the addition of blood sample to the receptacle. Alternatively or additionally, the lytic agent, (e.g., saponin) may be isolated from the resins, nutrient media, and anticoagulant prior to the addition of blood sample to the receptacle. Some or all of the antimicrobial-adsorbing resins may be isolated from the anticoagulant and/or the nutrient media prior to the addition of blood sample. The receptacle may be under negative pressure such that the blood sample fills the receptacle when connected with a standard fitting to a venous IV. A gas mixture in the receptacle may be optimized for aerobic microorganism growth. The nutrient media may be a tryptic soy broth. The resin may be magnetic. The resin may be supported on a solid substrate. The blood sample may be incubated under conditions promoting microorganism growth prior to the concentration step. Growth prior to the concentration step may be performed differently than the manner in which the concentration step is performed. Growth prior to the concentration step may be performed in a portable system. The pellet may be incubated under conditions promoting microorganism growth following the concentration step and prior to introduction into the calorimeter. The concentration step may be performed by centrifugation, filtration, flocculation, or magnetic separation. Centrifugation may be performed at a speed of 1,000×g-50,000×g or 1,000×g-10,000×g. The pellet introduced to the calorimeter may be ≤20, ≤10, ≤8, ≤6, ≤4, ≤2, ≤1, ≤0.5 mL in volume. Fresh nutrient media and/or agar may be introduced into the sample prior to sample introduction into the calorimeter. Following concentration, the pellet may be removed from the receptacle into which the blood sample was collected and transferred to a second receptacle. Isothermal or differential scanning calorimetry may be performed to determine microorganism growth. Isothermal calorimetry may be performed.

### Rapid Assessment of Microbial Growth by Calorimetry

As discussed above, some methods according to the present disclosure utilize isothermal or differential scanning calorimetry as a sensitive means for rapid detection of microbial growth. Positive growth may be determined based on absolute and/or relative heat flow from the sample under test. The method of heat flow used to determine positive growth may be different for different samples. Samples with growth periods beyond a threshold of 2, 3, 4, 5, 6, 7, 8, 9, 10 hours prior to the concentration step may have a different method of heat flow determination than samples with growth periods lower than this threshold. A sample may be loaded into the calorimeter upon processing readiness. A sample may be batched prior to loading into the calorimeter. A sample registering positive growth may be removed from the calorimeter on a sample-by-sample basis. A sample registering positive growth may be removed from the calorimeter on a batch basis. The secondary growth determination following sample removal may be performed after 2-48, 2-24, or 2-12 hours of incubation in the calorimeter. The incubation time in the calorimeter and the secondary growth method combined may be about 5 days. The supernatant of the sample following concentration may be incubated under conditions promoting microorganism growth. The supernatant may be measured for microorganism growth using at least one of optical absorbance, optical spectroscopy, optical microscopy, pH measurement, gaseous measurement, mass measurement, or electrical measurement. Supernatant growth measurement may be performed approximately in parallel with concentrated sample growth measurement. The result of the growth measurement from one or more of the concentrate and supernatant samples may be returned to the user. Microorganism identification and/or antimicrobial susceptibility testing and/or antimicrobial resistance determinations may be performed following the determination of positive growth. A cushioning fluid, defined as a water-miscible or water-immiscible fluid or solution with a density greater than that of a plurality of eukaryotic and prokaryotic cells, may be added to the receptacle prior to the concentration step. The cushioning fluid may be added directly prior to the concentration step. The cushioning fluid may be added to the receptacle simultaneously with the blood sample. The cushioning fluid may be comprised in the receptacle to which the blood sample is introduced. The method may be automated.

According to certain embodiments, a method for culturing blood samples suspected of comprising microorganisms may comprise contacting a blood sample with resins capable of adsorbing antimicrobial agents, performing at least one concentration step to concentrate microorganisms into a pellet separate from a residual supernatant, measuring the pellet for microorganism growth during incubation, and retaining a portion of the supernatant and any remainder of the pellet following the concentration step in a “backup” sample.

In some embodiments, a method for culturing blood samples suspected of comprising microorganisms may comprise contacting a blood sample with a resin capable of adsorbing antimicrobial agents, introducing a portion of the sample into an isothermal calorimeter, measuring heat flow from the sample to determine positive microorganism growth based on an absolute or relative signal, and if no growth is measured, removing the sample from the calorimeter and transferring the sample to at least one secondary growth determining systems.

Alternatively, or additionally, an automated method for determining microorganism growth in samples of human origin may comprise introducing the following components into a vessel prior to centrifugation: a sample of human origin, such as blood, cerebrospinal fluid, synovial fluid, plural fluid, pericardial fluid; one or more components capable of lysing eukaryotic cells; one or more barrier fluids, defined as a water-miscible or water-immiscible fluid or solution with a density and/or viscosity such that: it partitions below lysed blood cells during centrifugation; a plurality of lysed blood cells cannot enter the layer it forms; and microorganisms may enter the layer it forms during centrifugation; optionally, a cushioning fluid, defined as a water-miscible or water-immiscible fluid or solution with a density greater than that of a plurality of eukaryotic and prokaryotic cells; optionally, one or more anticoagulants; and optionally, one or more anti-foaming components; into a collection tube comprising the sample of human origin. The method may further comprise centrifuging the mixture under conditions suitable to enable microorganisms to enter the barrier fluid, removing the blood cell layer and introducing it into a nutrient media capable of supporting microorganism growth and performing one or more discrete or continuous monitoring methods for determining microorganism growth during incubation under conditions promoting microorganism growth, and introducing a plurality of the remaining microorganisms in the centrifuged vessel to a nutrient media capable of supporting microorganism growth and performing one or more discrete or continuous monitoring methods for determining microorganism growth during incubation under conditions promoting microorganism growth.

In certain embodiments, an automated method for determining microorganism growth in samples of human origin may comprise introducing a blood sample or treated blood sample into a vessel comprising a liquid or semi-solid filtering layer, centrifuging the mixture under conditions suitable to enable microorganisms to enter the filtering layer but that prevent a plurality of blood cells from entering the filtering layer, removing layer comprising blood cells above the filtering layer and introducing it into a nutrient media capable of supporting microorganism growth and performing one or more discrete or continuous monitoring methods for determining microorganism growth during incubation under conditions promoting microorganism growth, and introducing a plurality of the microorganisms present in the centrifuged vessel to a nutrient media capable of supporting microorganism growth and performing one or more discrete or continuous monitoring methods for determining microorganism growth during incubation under conditions promoting microorganism growth.

### Retention of Enriched and Depleted Sample Fractions

Those of skill in the art will appreciate that current laboratory standards for lysis-centrifugation, as described in e.g., U.S. Pat. No. 5,070,014, is to retain only pelleted material following centrifugation, potentially discards microorganisms from the original blood sample. This lowers positivity rates compared with continuous monitoring systems (Pohlman et al.) and led in part to the lysis-centrifugation technique being replaced by continuous monitoring systems in the mid-1990s.

To better understand the cause of the lower lysis-centrifugation positivity rates, the data in Tables 1-2 show the number of microorganisms in each volume fraction of a tube following performance of the lysis-centrifugation technique. These data demonstrate that while the majority of microorganisms are concentrated in the pellet, a significant percentage are retained in the supernatant and thus discarded following the original lysis-centrifugation procedure. Furthermore, the discarded percentage increases for more dilute samples, which is particularly detrimental if microorganism growth is to be performed from the time of collection because dilution into nutrient media is important for optimal growth.

Turning first to Table 1, samples of 10 mL lysed blood inoculated with E. coli were centrifuged according to lysis-centrifuge technique. Volumes of supernatant were removed and incubated overnight in TSB to reveal the presence of bacteria or plated on blood agar plates and incubated at 35° C. overnight. Presence of bacterial growth in TSB or individual CFUs were counted and recorded. Percent from the total CFU recovered per tube were calculated. Fractions in the table refer to volumes, in mL, beginning from the top or meniscus of the sample and working down. For instance, the 1-5 fraction comprised the first 5 mL of supernatant, fraction 6 comprised the next 1 mL, etc. Pellet refers to the pelleted material and excluding substantially all of the liquid phase. CFU Sum represents the total number of colony forming units (CFU) observed across all fractions.

TABLE 1
number of microbes in each volume fraction from
10 mL sample

| | Replicate 1 | | Replicate 2 | | Replicate 3 | |
|---|---|---|---|---|---|---|
| Fraction | CFU | % CFU | CFU | % CFU | CFU | % CFU |
| 1-5 | + | | + | | + | |
| 6 | 4 | 4.65% | 4 | 3.31% | 5 | 6.41% |
| 7 | 4 | 4.65% | 2 | 1.65% | 5 | 6.41% |
| 8 | 4 | 4.65% | 0 | 0.00% | 3 | 3.85% |
| 9 | 5 | 5.81% | 10 | 8.26% | 7 | 8.79% |
| 10 | 6 | 6.98% | 8 | 6.61% | 3 | 3.85% |
| Pellet | 63 | 73.26% | 97 | 80.17% | 55 | 70.51% |
| Total | 86 | 100% | 121 | 100% | 70 | 100% |

Table 2 summarizes data from 10 ml samples of lysed blood inoculated with E. coli and combined with of 40 mL TSB. Samples were centrifuged according to lysis-centrifuge technique. Volumes of 10 and 1 mL of supernatant were removed and centrifuged again to pellet bacteria. Volumes were plated on blood agar plates and incubated at 35° C. overnight. CFUs were counted and recorded. Percent from the total CFU recovered per tube were calculated.

TABLE 2
Number of CFU in each volume fraction after the lysis-
centrifugation technique

| CFU | Sam- | Sam- | Sam- | % CFU from Total | | | |
|---|---|---|---|---|---|---|---|
| ml | ple 1 | ple 2 | ple 3 | ml | Sample 1 | Sample 2 | Sample 3 |
| 1-10 | 8 | 8 | 14 | 1-10 | 4.47% | 4.57% | 7.41% |
| 11-20 | 17 | 9 | 12 | 11-20 | 9.50% | 5.14% | 6.35% |
| 21-30 | 12 | 10 | 10 | 21-30 | 6.70% | 5.71% | 5.29% |
| 31-40 | 12 | 6 | 8 | 31-40 | 6.70% | 3.43% | 4.23 |
| 41 | 0 | 1 | 1 | 41 | 0.00% | 0.57% | 0.53% |
| 42 | 3 | 1 | 2 | 42 | 1.68% | 0.57% | 1.06% |
| 43 | 0 | 0 | 2 | 43 | 0.00% | 0.00% | 1.06% |
| 44 | 3 | 0 | 1 | 44 | 1.68% | 0.00% | 0.53% |
| 45 | 2 | 1 | 1 | 45 | 1.12% | 0.57% | 0.53% |
| 46 | 3 | 2 | 1 | 46 | 1.68% | 1.14% | 0.53% |
| 47 | 3 | 2 | 0 | 47 | 1.68% | 1.14% | 0.00% |
| 48 | 5 | 3 | 2 | 48 | 2.79% | 1.71% | 1.06% |
| 49 | 5 | 2 | 5 | 49 | 2.79% | 1.14% | 2.65% |
| 50 | 28 | 11 | 16 | 50 | 15.64% | 6.29% | 8.47% |
| Pellet | 78 | 119 | 114 | Pellet | 43.58% | 68.00% | 60.32% |
| Total | 179 | 175 | 189 | Total | 100% | 100% | 100% |

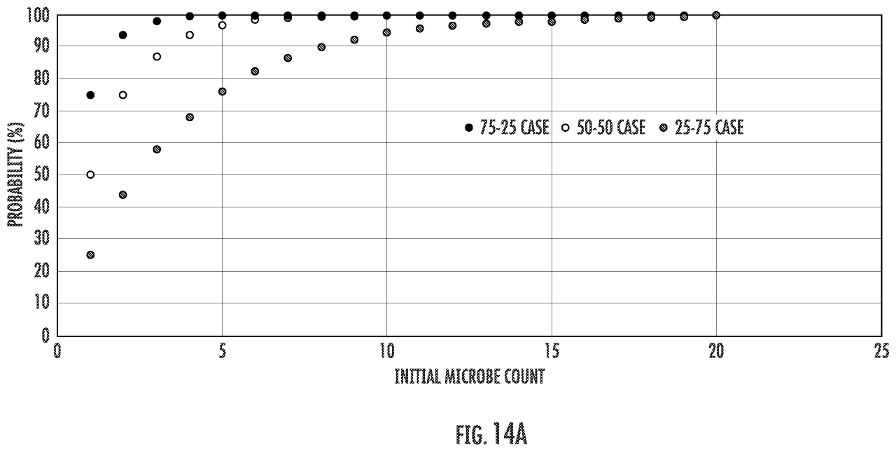

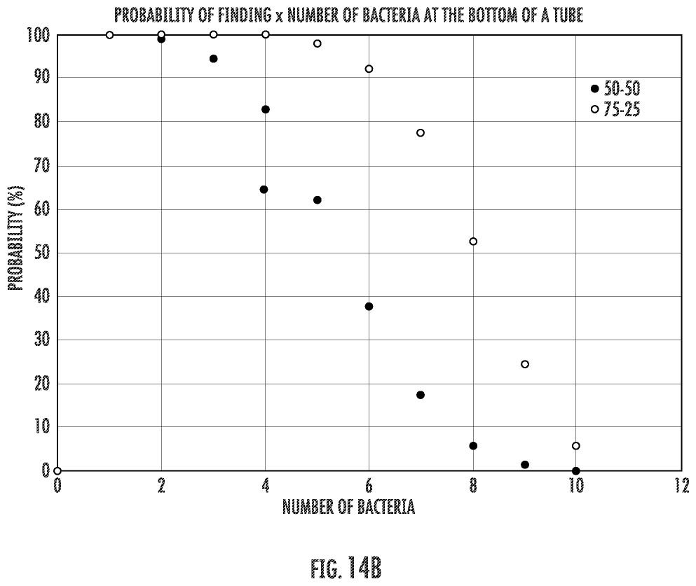

The presence of microorganisms in the supernatant can be most detrimental for performance when very few microorganisms are present in the initial sample. For example, in cases where only 1 microorganism is present per 10 mL sample draw, known by those skilled in the art to be a common occurrence, there is a substantial probability that this microorganism is not pelleted. Embodiments of the present disclosure in which the supernatant is cultured in addition to the pellet or concentrate advantageously increase the likelihood that microbes will be recovered from samples even at very low concentrations. For instance, without wishing to be bound by any theory, FIG. 14a depicts probabilities of capturing at least one bacterium in a concentrated fraction of a blood sample against the number of bacteria in the sample across three different rates of capture. In an instance where 75% of bacteria in the sample partition into the pellet, there is a comparatively high likelihood that the pellet will contain at least one bacterium even if there are as few as 2 or 3 bacteria in the sample. However, if the bacteria partition into the pellet less efficiently, e.g., at rates of 50% or 25%, then the probably that at least one bacteria will be captured in the pellet is correspondingly reduced, and the likelihood of a false negative result occurring increases, Similarly, FIG. 14b plots the probabilities of finding a given number of bacteria in a concentrated fraction when bacteria partition into that fraction at rates of 75% and 50%, and illustrates that the likelihood that a given percentage of microbes will be captured in the concentrated fraction is affected significantly by a fairly small reduction in the rate of partitioning.

To address the false-negative potential of samples with few microbes, sample preparation embodiments of this disclosure, variously, retain the non-concentrated fraction for analysis rather than disposing of it as is the current art-standard, and/or expose clinical samples to conditions that facilitate microbial growth before sample preparation, potentially increasing the number of replicating microbes in a sample to increase the likelihood that microbes will be captured in the concentrated fraction.

According to various embodiments of the present disclosure, blood samples or blood-derived samples are separated into microbe enriched and microbe depleted fractions. For simplicity, these fractions are typically referred to as “pellet” and “supernatant,” respectively. While this terminology reflects the suitability of centrifugation for achieving such separation, those of skill in the art will appreciate that separations can be also be achieved using filtration, affinity capture, size exclusion, and other methods which are also within the scope of this disclosure. Thus, the terms pellet, concentrate, enriched fraction or enriched portion, and like terms may be used interchangeably to refer to the concentrated fraction of a blood- or blood-derived sample, while the terms supernatant, depleted fraction, and like terms are used interchangeably to refer to the depleted fraction of a blood- or blood-derived sample.

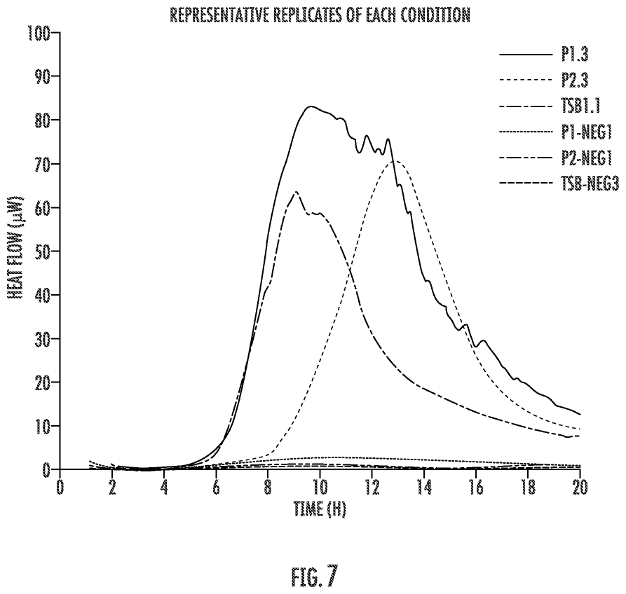

The concentrate, to which additional growth media may be added, may then be subjected to microorganism growth determinations by calorimetry. The data in FIG. 7 demonstrate the benefit of saponin lysis and concentration relative to concentration alone prior to calorimetry-based microorganism growth detection. The importance of performing the concentration step as well as the lytic step are further demonstrated in the data in Table 3. Blood samples from septic patients typically comprise 1-10 CFU/mL microorganisms, though as few as 1 CFU per 10 mL may be present. Since patients showing empiric signs of septicemia are typically placed on broad-spectrum gram-positive and gram-negative agents immediately after blood draw per standard care and broad-spectrum agents are typically given intravenously on 8-hour dosing cycles, an opportunity exists every 8 hours to alter therapy. As known to those skilled in the art, time is required for results to be transferred from the laboratory to the clinical floor thus there is an approximately 6 hour window for diagnostic testing results to alter broad-spectrum therapy.

The data in Table 3 demonstrate that calorimetry combined with lysis-centrifugation processing is advantageous for achieving a maximal TTD with calorimetry, approaching that of media alone. These data further demonstrate that, for a typical 10 CFU/mL blood sample, concentration of the sample by a factor of 10-20-fold is important for providing results within the timeframe for altering empiric therapy. These data further illustrate that calorimetry is able to detect growth from a variety of pathogenic microorganisms, and suggests that calorimetry can provide a level of sensitivity, as evidenced by TTD, that is greater than or equal to that of a standard OD660 measurement.

TABLE 3
Time to detection (TDD) of samples after lysis-centrifugation
technique by Calorimetry or optical density

| | Time to detection (hours) | | |
|---|---|---|---|
| Species | Calorimetry | OD660 | CFU/sample |
| E. coli | 6.2 | 8.14 | 13 |
| S. aureus | 5.55 | 7.80 | 11 |
| P. aeruginosa | 8.31 | 13.78 | 12 |
| A. baumannii | 6.41 | 10.86 | 26 |
| E. cloacae | 5.05 | 8.42 | 8 |
| E. faecium | 10.27 | 11.11 | 6 |
| S. pneumoniae | 5.69 | 8.14 | 13 |
| S. pyogenes | 5.56 | 8.19 | 23 |

In an embodiment, at least a portion of the concentrate, typically a loose pellet, is transferred to a separate receptacle, which may then be introduced into the calorimeter. This second receptacle may comprise fresh nutrient media and/or agar.

In some embodiments, the concentrate sample may be tested for microorganism growth by an optical technique, such as scattering or absorbance.

Samples may be loaded into the calorimeter on a random-access basis or may be batched for loading at discrete time intervals. Such time intervals may be every 1, 2, 3, 4, 5, 6, 7, or 8 hours. In an embodiment, samples in the calorimeter may be incubated isothermally under conditions promoting microorganism growth, between 31-39° C. In alternative embodiments, differential scanning calorimetry may be used within a range of 20-40° C. Sample mixing may also be performed mechanically, as known to those skilled in the art. In alternative embodiments the samples introduced to the calorimeter may have a nutrient agar present, such that solid-phase growth may occur. In alternative embodiments the samples introduced to the calorimeter may have nutrient agar present as well as nutrient broth, such that microorganisms may be growth in both solid- or liquid-phases.

In alternative embodiments, concentrate samples may be incubated under conditions promoting microorganism growth prior to introduction into the calorimeter.

In an embodiment, samples under test for microorganism growth in the calorimeter may reside within the calorimeter for periods of time less than the 5 days to determine a blood culture “negative” in standard clinical practice. Removed samples may be transferred to one or more additional growth determining mechanisms, such as optical, pH, gaseous or electrical. In this way, space in the calorimeter may be conserved and utilized for rapid results, whereas slower-growing samples may be measured using a lower-cost method.

In an embodiment, concentrate samples for which microorganism growth is detected may be removed from the calorimeter directly following positive growth determination or may continue incubating in the calorimeter for a defined period of time for sample batching purposes. Positive growth determinations may be based on absolute or relative heat flows. The data in Table 3 shows the relative TTDs for determining positive growth based on a relative heat flow of 10% compared with the peak heat flow and a relative value expressed as a percentage above the baseline. The determination of positive growth based on a relative heat flow above baseline may be advantageous if minimal growth has occurred prior to entry into the calorimeter. In cases where samples have undergone incubation prior to entry into the calorimeter, a pre-determined threshold may be utilized. These detection methodologies may be used in parallel, such that positive growth is registered by whichever threshold is met first.

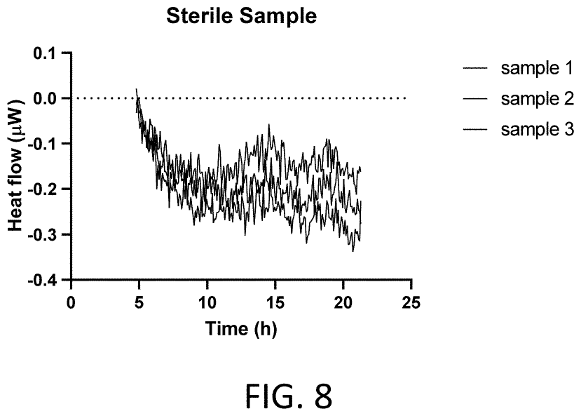

A key shortcoming of current pH-based systems is their inability to define a stable baseline because of continually-changing pH, which prevents sample incubation prior to entry into the systems. An important advantage of calorimetry following lysis-centrifugation is the stable baseline, defined as the ability to attribute heat flow above a pre-determined threshold to microorganism growth. This is illustrated in FIG. 8, which shows that non-microorganism-comprising samples do not register positive heat flows.

Certain embodiments of this disclosure also incorporate a staged analysis workflow in which samples under test for microorganism growth in the calorimeter may reside within the calorimeter for periods of time less than the 5 days to determine a blood culture “negative” in standard clinical practice. Removed samples may be transferred to one or more additional growth determining mechanisms, such as optical, pH, gaseous or electrical. In this way, space in the calorimeter may be conserved and utilized for rapid results, whereas slower-growing samples may be measured using a lower-cost method. In an embodiment, for the case of concentrate samples in which no microorganism growth is detected the samples may be removed from the calorimeter following a set period of time, which may be between 2-48 or 2-12 hours. The sample may then be separately measured for microorganism growth by a different method. This method may be any known to detect microorganism growth including, but not limited to, optical absorption, optical scattering, pH measurements, gas measurements, mass measurements, impedance measurements, etc. This design may be advantageous for preserving sample space in the calorimeter. In an embodiment, a baseline measurement with this secondary growth determination method is performed prior to the introduction of the concentrate sample into the calorimeter.

In an embodiment, in parallel with the concentrate being measured by calorimetry the supernatant may be separately measured for microorganism growth by a different method. This method may be any known to detect microorganism growth including, but not limited to, optical absorption, optical scattering, pH measurements, gas measurements, mass measurements, impedance measurements, etc. In an embodiment, this supernatant growth determination method is the same as the secondary growth determination method for concentrate samples. In an embodiment, a baseline for the supernatant growth determination method is performed prior to the concentration step.

For pediatric patients or other cases where smaller volumes of blood may be obtained from patients, similar methods to those described above may be performed, though the supernatant-comprising sample may have a lower volume.

In some embodiments it may be preferable to utilize one or more methods of optical detection, such as absorbance, transmittance, and scattering, for determining microorganism growth. In some embodiments these detection methodologies may be used in parallel with or in place of calorimetry. In some embodiments the separation step prior to detection may be designed to remove a plurality of mammalian cells. This may be advantageous for achieving a lower limit of detection (higher sensitivity) for optical detection and a more stable baseline for calorimetry, similar to the baseline provided by the selective mammalian cell lysis step. In these cases it may not be necessary to lyse cells prior to performing the separation step, thus it may be preferable not to perform a selective mammalian cell lysis step. In order to more completely separate microorganisms from mammalian cells, one or more liquid layers with densities greater than that of water may be utilized.

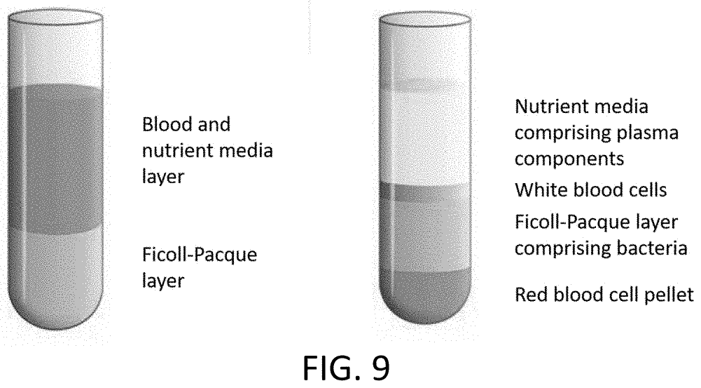

An exemplary illustration of the separation resulting from centrifugation following the layering of a blood sample above a Ficoll-Paque layer is shown in FIG. 9. Following centrifugation in which a non-lysed blood sample is layered above a Ficoll-Paque or similar layer, a plurality of less dense mammalian cells, such as platelets and white blood cells, will remain above the layer, whereas more-dense red blood cells will pellet. If microorganisms are present, the centrifugation time may be set such that remain suspended in the dense liquid layer, thereby enabling a portion of them to be separated from a plurality of the mammalian cells. Since some microorganisms may sediment while others may be trapped above the Ficoll-Pacque layer, the resulting red blood cell pellet and media/plasma/white blood cell layers may be combined to form the “backup” sample for microorganism growth detection. The microorganisms in the Ficoll-Pacque layer may be removed from a plurality of the Ficoll-Pacque, such as by dilution and/or subsequent centrifugation prior to the onset of optical detection and/or calorimetry for determining microorganism growth.

In alternative embodiments, two or more Ficoll-Pacque or similar layers with different densities may be layered above one another. By selecting an upper layer that microbes can easily sediment through during centrifugation and a lower layer that slows their sedimentation during centrifugation, microbes may preferentially be located at the interface between the layers following centrifugation. This may be advantageous for isolating microbes from mammalian cells that cannot easily sediment through the upper layer.

### Sample Collection and Handling

Continuous monitoring of blood cultures using platforms such as those described in U.S. Pat. No. 5,624,814 represent the current clinical microbiology laboratory state-of-the-art. These platforms gained favor over lysis-centrifugation techniques, such as the Wampole Isolator® (Alere Informatics, Inc., Charlottesville, VA) described in U.S. Pat. No. 4,164,449, because of improved reproducibility and human usability. In particular, the multiple manual steps required by the lysis-centrifugation technique introduced highly undesirable contamination and user-to-user variation.

However, continuous monitoring blood culture systems are characterized by three potential shortcomings that may delay results. First, these systems require bacteria to grow in the presence of bloodborne agents that may inhibit bacterial growth, such as white blood cells and platelets, cationic peptides, and intravenous antibiotics. Second, because of the CO2-based detection mechanisms employed by the leading systems, positive results are often not obtained until bacterial levels of 10⁷-10⁸ CFU/mL are achieved. Third, since continuous monitoring is required, in a consolidated healthcare system where bottles can be collected at sites that are geographically separated from the central clinical laboratory, incubation of the bottles cannot commence until they are loaded into the central laboratory's continuous monitoring platform. Thus, time waiting and in transport may be lost, as microbial growth may only occur once the sample is in the system, where is it held in conditions suitable for microbial growth.

Embodiments of this disclosure relate to multiple novel approaches for performing blood culture that speed time-to-positivity. The methods of this disclosure further enable incubation to commence at the site of collection and proceed during waiting and transport times prior to sample arrival in the central laboratory.

Some embodiments presented herein are based on the findings that a pellet produced from a sample by the lysis-centrifugation technique may be subsequently transferred to a single liquid or solid media to be observed for non-quantitative growth without any subsequent washing or removal of the cushioning fluid. These findings are surprising because they are contrary to literature teachings in three primary aspects. First, U.S. Pat. No. 3,928,139 and U.S. Pat. No. 4,141,512, in which Dorn introduced the lysis-centrifugation method following his development of a method to detoxify saponins disclosed in U.S. Pat. No. 3,883,425, teach that the two primary objectives of the method (1) are to achieve quantitative culture and (2) to enable a single blood draw to be cultured on multiple different solid nutrient agars. Second, U.S. Pat. No. 4,141,512 teaches that the cushioning agent important for achieving maximal bacterial retention should be selected such that it can be evaporated during solid-phase nutrient agar culture. Third, U.S. Pat. No. 5,070,014 teaches that the components necessary for effective lysis and separation for the lysis-centrifugation technique, saponin and sodium polyanethole sulfonate (SPS), are sufficiently toxic to microorganism growth at the concentrations present that they must be diluted following centrifugation. As a result, U.S. Pat. No. 5,070,014 teaches that solid phase culture must follow lysis-centrifugation processing and should utilize 6-inch, rather than standard 3-inch, petri dishes in order to more completely dilute the lytic components.

As is discussed in greater detail elsewhere in this specification, antimicrobial-adsorbing resins may be included in the vessel in which the blood sample is collected and the lytic agent is optionally present. Viewed in the context of minimizing time to result, the inclusion of such resins may be particularly advantageous because the time between sample collection and sample processing may be variable, owing to delays in transfer of the sample to the clinical laboratory, and the presence of adsorbent resins may abrogate the detrimental effects antimicrobials may have on microorganisms. This delay may be exacerbated in cases where satellite hospitals serve as collection sites for a centralized laboratory.

According to certain embodiments of this disclosure, nutrients that enhance microorganism growth, (e.g., growth media), may be included in the blood sample collection along with the lytic agent and/or adsorptive resin is optionally present. In this way, microorganism growth may be achieved prior laboratory processing (e.g., prior to the centrifugation step in sample processing). Growth may be further facilitated through sample incubation under conditions known to those skilled in the art to promote microorganism growth, such as a temperature of 33-40° C. and/or agitation during all or part of the interval between sample collection and arrival in the lab (e.g., during sample transport). If the sample is kept in these conditions, one or more lytic components may be added to and/or increased in concentration in the vessel prior to centrifugation. Such growth may be advantageous for increasing the number of microorganisms present in the sample prior to centrifugation and may further be advantageous for enabling growth during sample transport, especially in the case of centralized hub-and-spoke network designs.

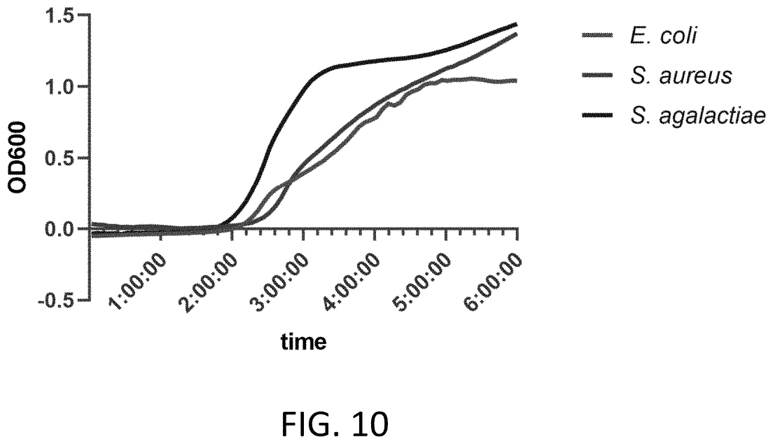

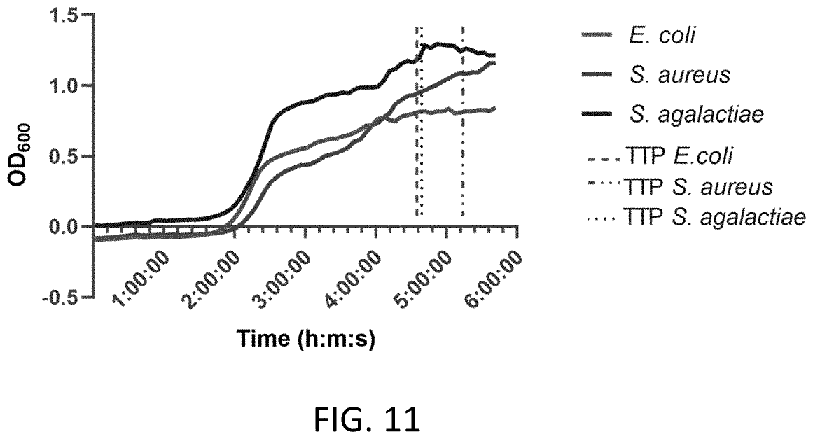

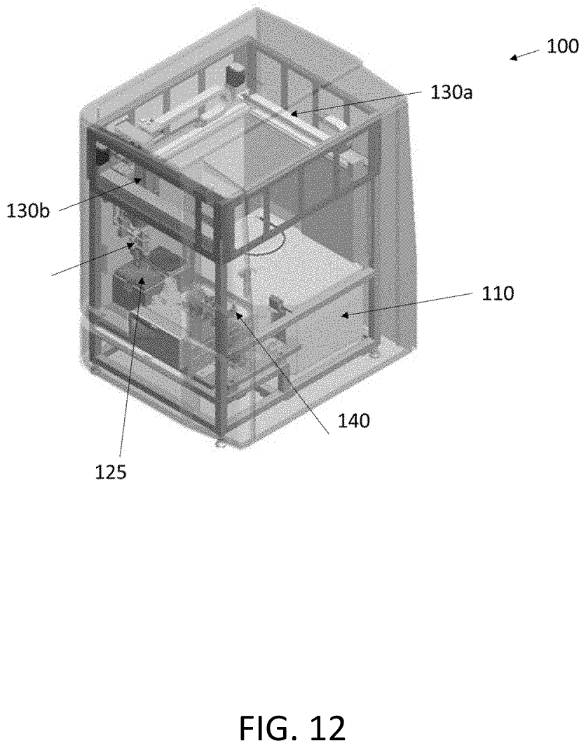

Here we show that microorganisms pelleted after use of the lysis-centrifugation technique and optionally without a cushioning fluid may be directly cultured in liquid nutrient media, without any additional dilution steps or with one or more wash steps (FIG. 10). Furthermore, we show that optical detection techniques, including but not limited to one or more of absorbance, nephelometry, fluorescence, luminescence, may be utilized to detect microorganism growth from these liquid cultures (FIG. 10). We additionally disclose that calorimetry may be used as a method for determining microorganism growth. Taken together, these approaches may offer faster times-to-positive growth identification than possible with continuous blood culture monitoring systems (FIG. 11). These approaches may further enable sample incubation to begin at the site of collection, directly following lysis-centrifugation processing. This is contrasted with current continuous monitoring systems, such as U.S. Pat. No. 5,624,814, in which the plurality of incubation must occur while the bottle resides in the machine due to detection limitations, potentially requiring long wait times before incubations can begin for samples collected remotely. These limitations stem from the nature of detection used in these instruments. In order to differentiate CO₂/acidification produced by bacteria from blood cells, the instruments scan for the second derivative of CO₂/acidification to observe where there is an increase in slope above baseline. This corresponds to bacterial CO₂/acidification dominating that of the blood cell baseline. At this point, the bottle is called positive. It is essential the bottle be present in the machine during this time because CO₂/acidification rates are variable sample-to-sample and CO₂/acidification will reach a maximal rate followed by falling back to baseline. Thus, if the window during acceleration of CO₂/acidification is missed (if the bottle is not in the machine during this time), a false negative may be interpreted.

Since automation is important for minimizing user-to-user variation, we further describe a system capable of centrifuging the initial lysis-centrifugation vial, removing and discarding the supernatant following centrifugation, adding a nutrient growth media, and optionally transferring the pellet to a new tube. FIG. 12 shows a system 100 for automated preparation of samples according to embodiments of this disclosure. System 100 comprises a centrifuge 110 capable of spinning samples up to ˜6,000×g; an automated liquid handler 120 with disposable pipette tips 125 capable of adding and removing fluids; a gripper capable of holding, moving, and releasing tubes; a gripper (which may be the same as the previous) capable of uncapping tubes; and a 3-axis gantry 130a, b capable of enabling the liquid handler and gripper(s) to reach all points on the deck necessary for processing. In particular, the system in FIG. 12 combines the liquid handler 120 and gripper onto a single x,y-gantry 120a, each with an individually addressable z-gantry 120b, and further utilizes a single gripper for all movements, including de-capping. This system may also have utility for separating microorganisms from blood cultures to enable further sample processing, such as microbial identification or antimicrobial susceptibility testing, as well as one or more reservoirs 140 for consumables used during sample processing.

This disclosure also encompasses, in various embodiments, methods for microorganism growth detection in the presence of remaining eukaryotic cells and cell fragments following lysis-centrifugation. These methods may be used in parallel or sequentially and may give Gram typing information in addition to positivity. In addition, one or more microorganism genera or species may be determined by PCR or similar genetic methods.

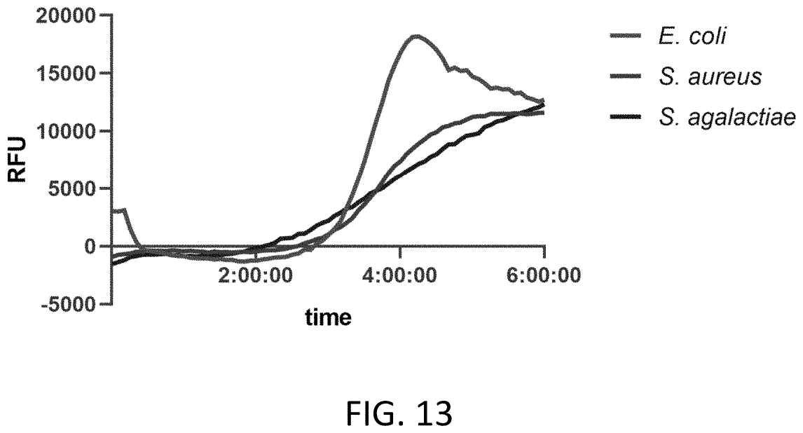

FIG. 10 showed the use of optical density readings at 600 nm to determine microorganism growth following lysis-centrifugation. Metabolic probes known to be reduced by microorganisms may also be used, such as resazurin, which may be coupled with one or more electron transport agents, such as methylene blue and 1-methoxy-5-methylphenazinium methyl sulfate (FIG. 13). Pre-fluorophores that are known to produce fluorescent products after undergoing one or more enzymatic reactions may also be used. These may include, but are not limited to, carboxylated fluorescein diacetate (esterases), 4-methylumbelliferyl phosphate and 6,8-difluoro-4-methylumbelliferyl phosphate (phosphatases), 4-Methylumbelliferyl-betaD-glucuronic acid dihydrate or Resorufin-β-D-glucuronic acid methyl ester (galactosidase), L-leucine-7-amido-4-methylcoumarin hydrochloride (peptidases). Fluorophores known to increase fluorescence upon specific binding or intercalation, such as to DNA or membranes, may also be used. These may include, but are not limited to, SYTO, Hoescht, YOYO, DiYO, TOTO, DiTO, 4′,6-diamidino-2-phenylindole, 7-aminoactinomycin, PMF, and other live/dead staining probes.

Probes capable of detecting macromolecules and macromolecule biosynthesis specific to bacteria may be particularly advantageous. US20150191763A1 disclosed D-amino acid-coupled fluorophores capable of being covalently bonded into bacterial peptidoglycan during microorganism growth. However, the measurement technique requires extensive washing prior to signal interrogation to ensure non-incorporated fluorophore-coupled probe is removed. Here we introduce the use of fluorescence polarization (anisotropy) to measure D-amino acid-coupled fluorophore incorporation into growing peptidoglycan chains. This technique takes advantage of the fact that unbound probes are <1,000 Daltons, whereas bound probes are effectively >>10,000 Daltons due to the crosslinked nature of peptidoglycan. Fluorescence polarization may also be interrogated for self-quenching, as known for homodimers. Since fluorophores with long lifetimes are advantageous for fluorescence polarization, we further introduce the concept of a D-amino acid-coupled pre-fluorophore, which can undergo multiple reactions within microorganisms. Enzymatic reactions convert the probe to its fluorescent form and its D-amino acid conjugation enables its incorporation into peptidoglycan. For example, a diacetate carboxyfluorescein may be coupled to D-lysine. This scheme may be particularly advantageous for two reasons. First, the literature teaches that charged molecules, such as fluorescein, cannot easily penetrate microorganisms, decreasing labeling efficiency. Second, the pre-fluorophore may reduce the fluorescent background. A two-D-amino acid-probe system may also be used to enable peptidoglycan measurements without centrifugation. In this case, Forster resonance energy transfer (FRET) may be utilized. In particular, it may be advantageous to utilize time resolved FRET (TR-FRET), such as can be achieved using lanthanide chelates, such as europium or terbium cryptates. In this case, a TR-FRET signal will be observed if a donor lanthanide chelate lies within a Forster radius (typically ˜1-10 nm) of a small-molecule acceptor (such as, but not limited to, Cy5 or AlexaFluor 647 for europium cryptate or Lance terbium or the same or fluorescein or AlexaFluor 488 for Lumi4-terbium).

A similar TR-FRET methodology may be utilized to create a surface-binding donor/acceptor pair. Surface binding may be achieved with cationic europium cryptate chelates, which may be paired with similarly cationic organic fluorophores to enable TR-FRET. Similarly, surface-associating probes may be interrogated with fluorescence polarization.

Calorimetry may also be used for detecting microorganism growth. This method may be advantageous over optical methods in that probes may not be required and that it may be less sensitive to lysed cells and cellular debris. Isothermal calorimetry is preferred over differential scanning calorimetry. Systems enabling multiple samples to be run in parallel, such as the TA Instruments (Wakefield, Mass.) model IV-48, the Omnical (Stafford, Tex.) Insight, or the Symcel (Solna, Sweden) celScreener, are preferred.

### EXAMPLES

### Example 1

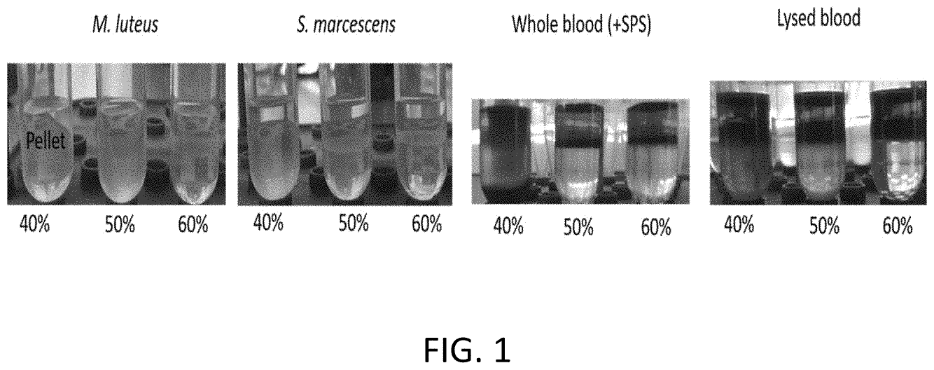

In order to identify suitable density layer configurations for separation and/or enrichment of microbial cells in blood samples, samples were prepared using purchased pre-made sucrose solutions at 40, 50 and 60%, 1.5 mL of which were added to round-bottom 10 mL centrifuge tubes. 1 mL of one of the following microbial suspensions was then added atop the sucrose layer: a suspension of M. luteus in saline of optical density at 600 nm (OD600) of ≥2.0, a suspension of S. marcescens in saline of OD600≥2.0, whole blood (+SPS) or lysed blood treated with lysis buffer (from Isolator10 tube- Wampole/Alere). Each tube was then centrifuged at 2370×g in a swinging bucket rotor for 30 min with the break set to 0 and the temperature to 30° C. A picture of each tube was taken using a smartphone camera following centrifugation. The images are shown in FIG. 1. Blood cells were found to enter the 40% sucrose layer but not the 50% and 60% sucrose layers. For lysed blood samples, lysed hemoglobin entered the 40% and 50% sucrose layers. The M. luteus and S. marcescens samples entered all sucrose solutions, with penetration depth inversely related to sucrose concentration. Note the 40% M. luteus sample pelleted and the 60% S. marcescens sample is in a band that lies within the sucrose layer (just below the top boundary).

### Example 2

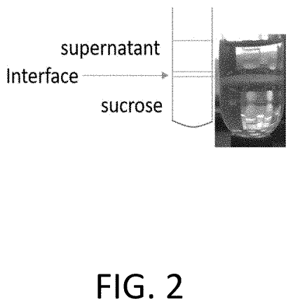

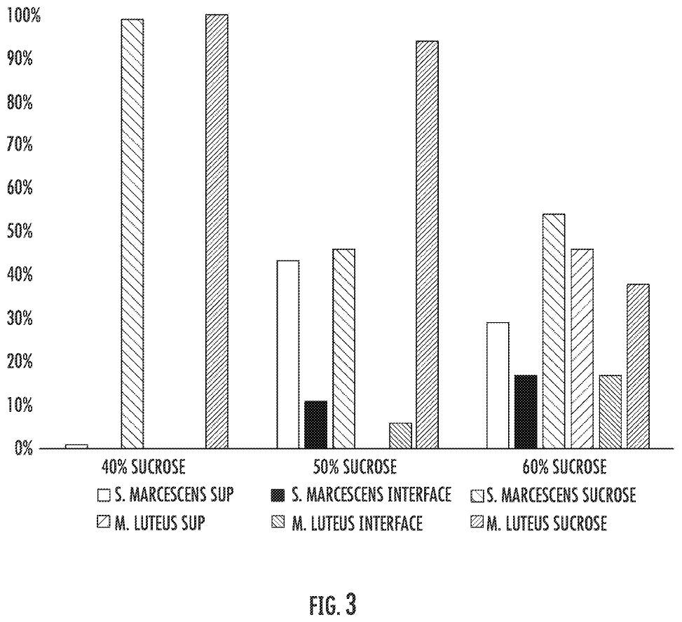

The partitioning of microbes from whole lysed blood samples into sucrose layers of varying density was examined further by preparing separate saline stock solutions of M. luteus and S. marcescens to OD600=0.1. These solutions were diluted to a final concentration of 100 CFU/mL in lysed whole blood lysed using the Isolator10 tube (Wampole/Alere). The resulting mixture was then layered on top of 1.5 mL sucrose (40, 50 or 60%) purchased commercially and centrifuged at 2370×g in a swinging bucket rotor for 30min with the break set to 0 and the temperature to 30° C. Quantitative culture was performed overnight using blood agar plates in sequential 10-fold dilutions. For each sample the following fractions of the centrifuged tubes were plated for quantitative culture: 1 mL supernatant (“sup”), 200 uL “interface” layer, 1.3 mL sucrose “filter” density layer. The layers are depicted in FIG. 2. As seen in the quantitative culture data in FIG. 3, the percentage of microorganisms entering the filter layer is roughly inversely proportional to the sucrose concentration.

### Example 3

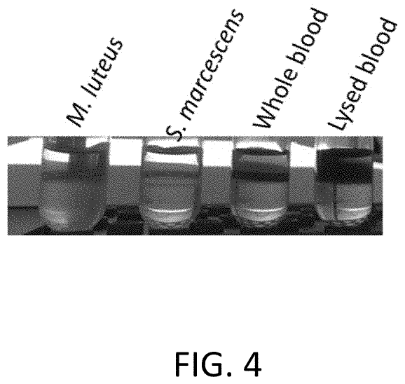

A solution of 50% sucrose (w/v, Sigma) and 1.5% gelatin (porcine, Sigma) was made, warmed to 42° C., and 1.5 mL was added to 10 mL round-bottom centrifuge tubes. The solution was gelled overnight at 4° C., and 1 mL of one of the following suspensions was then added atop the gel layer: a suspension of M. luteus in saline of optical density at 600 nm (OD600) of ≥2.0, a suspension of S. marcescens in saline of OD600≥2.0, whole blood (+SPS) or lysed blood treated with lysis buffer (from Isolator10 tube- Wampole/Alere). Samples were centrifuged at 2370×g in a swinging bucket rotor for 30 min with the break set to 0 and the temperature to 30° C. A picture of each tube was taken using a smartphone camera following centrifugation, shown in FIG. 4. As the figure demonstrates, M. luteus and S. marcescens organisms entered the filter layer while the whole and lysed blood did not.

### Example 4

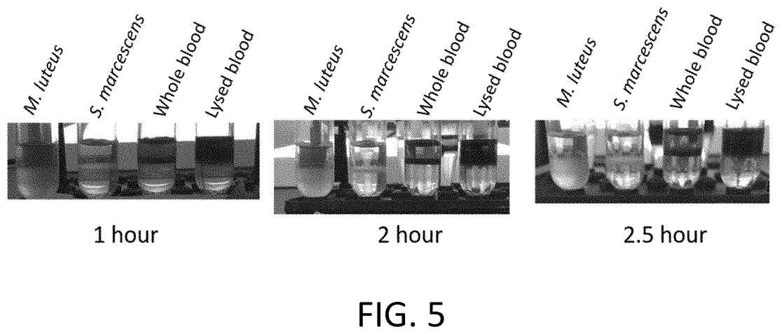

The same procedure as in Example 3 was followed with the exception that centrifugations of 1, 2, and 2.5 hrs were performed. The S. marcescens band is seen to progress further into the filter layer with increased centrifugation times, whereas the whole and lysed blood continues not to penetrate the layer, as seen in FIG. 5.

### Example 5

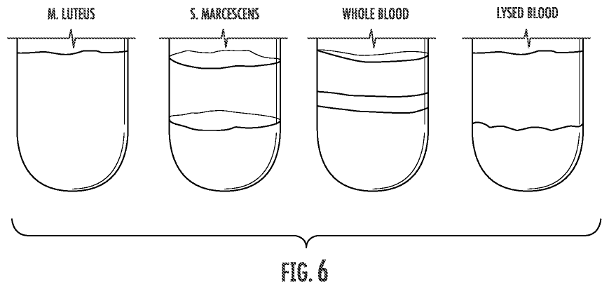

The same procedure as in Example 3 was followed with the exception that the sucrose-gelatin solution was warmed to 42° C. prior to centrifugation. The M. luteus and S. marcescens organisms are seen to enter the filter layer, as is hemoglobin lysed from red blood cells, FIG. 6. The blood cells are observed to reside above the filter layer.

### Example 6

In order to compare the performance of calorimetry versus absorbance-based growth determinations, whole blood was collected in vacutainer tubes containing sodium polyanethole sulfonate (SPS) and then processed by lysis centrifugation following the manufacturer's protocol (Isolator 10, Alere). Lysed blood samples were combined with tryptic soy broth (TSB; Sigma), and inoculated with Escherichia coli to a final concentration of 100 CFU/mL. Processed blood samples were placed into a plate reader (Biotek) or a calorimeter (Symcel) and incubated at 37° C. Time to detection (TTD) was determined based on a percentage increase in optical density (at 600 nm) or heat generation over baseline. Representative TTD for triplicate samples is shown in Table 3, presented in the section titled “Retention of Enriched and Depleted Sample Fractions” above.

### Example 7

10 mL of lysed blood inoculated with E. coli combined with of 40 mL TSB. Samples were centrifuged according to lysis-centrifuge technique. Volumes of 10 and 1 mL of supernatant were removed and centrifuged again to pellet bacteria. Volumes were plated on blood agar plates and incubated at 35° C. overnight. CFUs were counted and recorded. Percent from the total CFU recovered per tube were calculated. Data are presented in Table 2, in the section titled “Retention of Enriched and Depleted Sample Fractions” above. Taken together, these data indicate that, following centrifugation of a lysed blood sample, microbes become concentrated in the pellet but can be found, in many cases, throughout the supernatant as well. The data also indicate that sufficient numbers of microbes may remain in the supernatant and may support microbial growth determinations.

### Example 8

Whole blood was collected in vacutainer tubes containing SPS. A portion of blood was processed by lysis centrifugation. Whole blood (P2) and a “processed” sample comprising the pellet from a blood sample subjected to lysis-centrifugation (P1) were combined with tryptic soy broth (TSB) and inoculated with E. coli to achieve a final concentration of 100 CFU/mL. Inoculated blood samples and TSB media sample were placed in a calorimeter (Symcel) and incubated at 37° C. Data for representative samples are shown in FIG. 7. Negative controls of uninoculated P1 media are plotted in gray in FIG. 8. Together, these data show that bacterial growth, evident as positive and/or increasing heat flow, can be detected in both whole blood and lysed-centrifuged blood (FIG. 7), while whole blood alone, and processed samples without bacteria, do not have positive heat flows (FIGS. 7 and 8). These data also indicate that positive heat flows can be used as a basis for distinguishing positive samples from negative samples by calorimetry.

### Example 9

Whole blood was collected in vacutainer tubes containing SPS. A 2 mL aliquot of the sample was spiked with 1000 CFU/mL E. coli bacteria (ATCC) and subsequently mixed 1:1 with TSB nutrient media under aseptic conditions. This was then aseptically layered above a 3 mL volume of sterile Ficoll-Pacque in a 12 mm centrifuge tube. The tube was spun at 400×g for 35 minutes with no brake. The resulting tube, illustrated schematically in FIG. 9, was then processed under aseptic conditions as follows. The supernatant layer comprising all layers above the Ficoll-Pacque was pipetted into a separate tube (tube A) and vortexed. The Ficoll-Pacque layer was then drawn up with a pipette, taking care to stay approximately 2-3 mm away from the red blood cell pellet interface, and transferred into a separate tube (tube B) and vortexed. The red blood cell pellet interface was washed with 500 μL of TSB twice and transferred into a separate tube (tube C). The remaining pelleted was vortexed after the addition of 1 mL of TSB (tube D). All tubes were plated for quantitative culture by drawing 500 μL from each tube onto nutrient agar comprising sheep blood and grown overnight. The calculated bacteria count based on the volume of each layer from the averaged results of the quantitative culture performed in duplicate are shown in Table 4. The majority of bacteria are found in the Ficoll-Pacque layer. The number of bacteria in the pellet may be reduced by using a longer Ficoll-Pacque length, whereas the number of bacteria in the supernatant may be reduced by increasing centrifugation time or speed.

TABLE 4
CFU in different layers following centrifugation

| Layer | Calculated bacteria count (CFU) |
|---|---|
| Supernatant | 312 |
| Ficoll-Pacque | 1278 |
| Interface | 41 |
| Blood pellet | 417 |

### Example 10

The same procedure in Example 9 was utilized with the exception that the blood was lysed with saponin as in previous examples prior to its spiking with E. coli and 1:1 mixing with TSB. This same procedure was performed on two samples, one of which was spun at 400×g (Sample A) and the other at 800×g (Sample B). For sample A, only 33% of the bacteria were present in the Ficoll-Pacque layer following centrifugation, whereas for sample B, approximately 70% of the bacteria were present in the Ficoll-Pacque layer.

### Example 11

To demonstrate the feasibility of the lysis-centrifugation sample preparation methods described in this disclosure, these methods were modeled by spike-in experiments in which known quantities of bacteria were inoculated in normal blood cultures, which were then processed by lysis-centrifugation methods as described herein. Bacterial strains used in this study were Escherichia coli ATCC 25922, Staphylococcus aureus ATCC 29213, and Streptococcus agalactiae SD-1234. Bacteria were cultured on tryptic soy agar with 5% sheep's blood (Northeast Laboratory) at 35° C. to obtain isolated colonies. Blood collected in SPS vacutainer tubes (BD364960) was inoculated with bacterial saline suspensions to an estimated final concentration of 1×10⁵ CFU/mL (based on OD600 measurements of diluted colonies) or with saline as a sterile control. Inoculated blood (10 mL) was transferred to Isolator 10 tubes containing lysis buffer (Alere). Isolator tubes were processed according to manufacturer's instructions. Briefly, tubes were inverted 5 times to mix contents and centrifuged at 3000×g for 30min. The isostat system (Alere) was used to remove the supernatant and the pellet was transferred to a sterile reservoir. The pellet was diluted 1:4 with tryptic soy broth (TSB; Sigma-Aldrich) prepared according to the manufacturer's instructions into black 96-well flat, optical bottom plates (Thermo scientific). Plates were incubated at 37° C. in a Bio-Tek plate reader with orbital shaking. Bacterial growth was measured at regular intervals by optical density (OD) absorbance readings at 600 nm or by alamar blue flourescence at 560/590 (ex/em). The average OD600 relative to the sterile control is graphed over time (hours) in FIG. 10.

### Example 12

To confirm that the methods of this disclosure can yield time-to-positive performance similar to current industry standards using a widely used commercial blood culture vial, samples were prepared substantially as described in Example 11 with the exception that, in parallel with introducing bacteria-spiked blood samples into Inoculator 10 vials, the same quantity of bacteria were inoculated into 10 mL of blood directly collected into BACTEC standard aerobic vials. These were then placed into a BACTEC 9050 continuously monitoring system according to the manufacturer instructions. The time to positivity (TTP) defined as the time from initial bacterial incubation until the BACTEC 9050 positive blood culture signal was recorded, and is graphed over time in FIG. 11.

### Example 13

In order to verify the lysis-centrifugation methods of this disclosure were applicable to fluorescence-based methods for monitoring microbial growth, samples were prepared substantially as described in Example 11 with the exception that the TSB was supplemented with alamar Blue (ThermoFisher), which served as a growth indicator probe and that bacterial growth was measured at regular intervals by fluorescence readings at 560/590 (ex/em) and presented in FIG. 13.

### Example 14

In order to confirm that adsorptive resins could be used to remove antimicrobial agents in sampled patient blood in a manner that was compatible with the lysis-centrifugation and calorimetric growth measurement methods described herein, blood was drawn from healthy donors into Bactec aerobic plus resin bottles and inoculated with bacteria as indicated. One set was inserted into the Bactec 9000 series for growth detection by the Bactec routine clinical method. The remaining bottles were processed by a method within the scope of the present disclosure, referred to in Table 5 as “L-C-C method”: Blood was lysed by adding lysis media to the bottles. The liquid blood culture was separated from the solid resins using a needle syringe. The sample was centrifuged 3000×g for 15min and supernatant was removed. Growth media was added to the blood pellet and then samples were placed in the calorimeter for detection of bacterial growth. The supernatant fraction was also incubated and the method according to the present disclosure includes growth detection from the calorimeter (rapid) and supernatant fractions. Data are presented in Table 5, below, and expressed as the fraction of total bottles with positive blood cultures. As the table indicates, the L-C-C method of the present disclosure provide positive results that are comparable to, and in some cases appear superior to, the current routine clinical method.

TABLE 5
Fraction of Positive Cultures Observed using Routine and
L-C-C Methods

| | | Fraction of Positive Cultures from Bactec | |
|---|---|---|---|
| | | Aerobic Plus bottles (#pos/total) | |
| | CFU/ | Bactec routine clinical | |
| | bottle | method | L-C-C Method |
| E. coli | 1 | 3/5 | 2/5 |
| K. pneumoniae | 1 | 5/6 | 6/8 |
| E. feacalis | 2 | 2/8 | 7/8 |

The effect of adsorptive resins on the time to result was also examined. Table 6 shows that the average time to result for E. coli samples achieved using the L-C-C method is similar whether or not resins are present.

TABLE 6
Average Time to Detection (TTD) With and Without Adsorptive Resins

| | Average TTD (H) |
|---|---|
| No resins present | 6.83 |
| Resins present | 7.12 |

### Example 15

Those of skill in the art will appreciate that a period of time may elapse between the collection of a clinical blood sample and the processing and culturing of that sample. This may extend the time required after the collection of the sample for a clinically meaningful result to be returned, and/or increase the potential for false negative results from samples with relatively low numbers of microbes, as collected samples are generally not stored in conditions that facilitate microbial growth until processing begins. Certain sample collection and processing methods of this disclosure involve maintaining blood samples under conditions that facilitate microbial growth, e.g., above ambient temperatures, within a controlled atmosphere, with added nutrients and/or media components that support microbial growth, and/or agitated rather than left still. These approaches are collectively referred to as “pre-incubation.” In addition, embodiments of this disclosure employ fractionation of samples into more concentrated (e.g., pellet) and less concentrated subsamples; both fractions are then retained, with the objectives of making a rapid assessment of positivity (from the concentrated fraction) and decreasing the likelihood of a false negative result (by interrogating the less concentrated fraction over a longer interval of time). To demonstrate that the pre-incubation and fractionation approaches described herein can improve microbial detection—as measured by false-negative performance and by microbial growth—blood was drawn from healthy donors into Bactec bottles and inoculated with very low (1-3 CFU per sample) numbers of the bacterial species indicated in Table 7. Blood was lysed using lysis media. The sample was centrifuged 3000×g for 15 min and supernatant was transferred to a new tube. Samples were then divided into two subsamples: A “rapid fraction” in which growth media was added to blood pellet and then the processed samples were placed in the calorimeter for detection of bacterial growth; and a “supernatant fraction” comprising the supernatant was also incubated for detection of bacterial growth. Following interrogation of the rapid and supernatant fractions, the presence of bacteria in tested samples was verified by agar plating.

As Table 7 shows, test samples averaged only 1-3 CFU per sample (labeled “Average CFU”). In this setting, examination of rapid fractions alone (labeled Rapid (+) in the table) unsurprisingly resulted in a small fraction of false negative results. However, when both rapid and supernatant fractions were examined, growth was detected in at least one fraction of each sample (labeled “Rapid (+) OR Supernatant (+)”). These results indicate that the likelihood of false negative results from very low CFU samples can be reduced by the retention and interrogation of both concentrated and depleted fractions according to the embodiments of this disclosure.

TABLE 7
Detection of bacterial growth in rapid and supernatant fractions

| | | Detection of bacterial | | | | |
|---|---|---|---|---|---|---|
| | | growth in (# of samples): | | | False | |
| | | | Rapid (+) | | Negative Rate | |
| | | | AND/OR | total | | Rapid plus |
| | average | Rapid | Super- | bottles | Rapid | Super- |
| | CFU | (+) | natant (+) | tested | only | natant |
| E. coli | 1 | 5 | 5 | 5 | 0/5 | 0/5 |
| E. faecalis | 2 | 5 | 7 | 7 | 2/7 | 0/7 |
| K. pneumoniae | 1 | 18 | 25 | 25 | 7/25 | 0/25 |
| P. aeruginosa | 3 | 11 | 11 | 11 | 0/11 | 0/11 |
| P. mirabilis | 1 | 3 | 6 | 6 | 3/6 | 0/6 |
| S. aureus | 1 | 20 | 26 | 26 | 6/26 | 0/26 |

To model the impact of pre-incubation after sample collection on the rate of false-negative results in the rapid fraction, blood was drawn from healthy donors into Bactec bottles and inoculated with bacteria as indicated above. Samples were either directly processed (including the lysis step) or incubated for 3 hours prior to processing. Each sample was centrifuged 3000×g for 15 min and supernatant was transferred to a new tube. For rapid detection, growth media was added to blood pellet and then the rapid detection samples were placed in the calorimeter for detection of bacterial growth. The supernatant fraction was also incubated for detection of bacterial growth. The percent of samples that were negative in the calorimeter but had growth in the supernatant fraction are indicated as % of false negatives in rapid detection fraction. As Table 8 indicates, the percent of false negative results among rapid-detection samples only was reduced significantly when pre-incubation was used; in fact, no false negative results were observed when pre-incubation was performed.

TABLE 8
Percent of False Negative Results in Rapid Fractions from Directly
Processed and Pre-Incubated Samples

| | | % of false negatives in rapid | |
|---|---|---|---|
| | | detection fraction | |
| | average CFU | Directly processed | Pre-incubation |
| S. aureus | 1 | 23 | 0 |

### Example 16

To confirm that the methods of the present disclosure are viable for clinical sample preparation and analysis, the performance of a method according to an embodiment of this disclosure was compared to the performance of commercially available industry standard blood culture processing systems. Blood was drawn from healthy donors into Bactec aerobic bottles and bacteria inoculated into the bottles. One set of Bactec bottles were inserted into the Bactec9000 series (Becton-Dickinson, Sparks Md.) for Bactec detection of growth. The remaining Bactec bottles were processed according the methods of the present disclosure: blood was lysed using lysis media. The sample was centrifuged 3000×g for 15min and supernatant was removed. Growth media was added to the blood pellet and then samples were placed in the calorimeter for detection of bacterial growth. The supernatant fraction was also incubated, and results from both the rapid and supernatant fractions were collected. Data from samples prepared and analyzed according to the method of this disclosure are denoted in Table 9 as “New” and compared to the performance of the Bactec analyzer for the microbes listed. As the table indicates, the “New” method of this disclosure achieved performance that was similar to, and in some cases superior to, the performance achieved by the Bactec analyzer.

TABLE 9
Positive and Total Samples Tested Using Bactec and New Methods

| | | Bactec | | New |
|---|---|---|---|---|
| | Bactec | Total | New | total |
| | postive | Tested | Positive | Tested |
| E. coli | 6 | 16 | 8 | 21 |
| E. faecalis | 8 | 8 | 7 | 8 |
| K. pneumoniae | 9 | 16 | 25 | 32 |
| P. aeruginosa | 13 | 13 | 21 | 24 |
| P. mirabilis | 7 | 8 | 6 | 8 |
| S. aureus | 14 | 15 | 34 | 49 |

In order to confirm that the success of the “New” method described could be replicated without the use of a calorimeter, the method was adapted to use a pH-based colorimetric method for detection of microbial growth (BacT/Alert, BioMeriux USA, Durham, N.C.). Blood was drawn from healthy donors into BacT Alert bottles and inoculated with bacteria as indicated. Bottles prepared according to routine clinical standards were inserted into BacT Alert for detection. Remaining bottles were processed according to the “New” method as follows. Blood was lysed using lysis media. The sample was centrifuged 3000×g for 15 min and majority of supernatant was removed and inserted into the BacT Alert for detection. Results for the standard clinical preparation and the “New” method are presented in Table 10. As the table indicates, both methods correctly identified the test cultures as positive across two replicates.

TABLE 10
Performance of Non-Calorimetric Methods of this disclosure vs.
Industry Standard

| | | % Positive samples | |
|---|---|---|---|
| | | Lysis- | Routine culture |
| | CFU/bottle | centrifugation | method |
| S. aureus | 4.2 | 100 | 100 |
| E. coli | 4 | 100 | 100 |
| P. aeruginosa | 7.8 | 100 | 100 |
| E. faecalis | 1 | 100 | 100 |
| K. pneumoniae | 1 | 100 | 100 |
| P. mirabilis | 9 | 100 | 100 |

However, as Table 11 shows, times to detection using the “New” non-calorimetric method was generally shorter than those achieved using the standard clinical preparation.

TABLE 11
Time to Detection for “New” and Routine Clinical Samples

| | | TTD (H) | |
|---|---|---|---|
| | | Lysis- | Routine culture |
| | CFU/bottle | centrifugation | method |
| S. aureus | 4.2 | 10.8 | 12.24 |
| E. coli | 4 | 10.92 | 11.76 |
| P. aeruginosa | 7.8 | 15.12 | 16.2 |
| E. faecalis | 1 | 12 | 12.96 |
| K. pneumoniae | 1 | 13.2 | 11.76 |
| P. mirabilis | 9 | 13.2 | 18.48 |

### Example 17

Serial dilutions of E.coli were made in TSB for different CFU/ml concentrations. Different volumes of these dilutions as indicated were added to TAM IV (TA instruments) or Calscreener (Symcel) calorimeters. TAM IV samples were either 4 ml or 20 ml glass vials and Calscreener samples were 0.5 ml plastic inserts. TA instruments and Symcel samples each contained a calculated 50 CFU/sample and 5 CFU/sample, respectively. Samples were incubated in the calorimeter at 37 C and heat flow over time was recorded. TTD was determined as the time that heat flow exceeded 10 μW from the baseline.

TABLE 12
Volume invariancy of time to detection for calorimetry

| | TTD (H), 10 μW threshold | | | |
|---|---|---|---|---|
| | TA instruments | | | |
| Sample | (50 CFU/sample) | | | |
| concentration | volume | | volume | |
| (CFU/ml) | (mL) | TTD | (mL) | TTD |
| | | | Symcel (5 CFU/sample) | |
| 1.00E+02 | 0.5 | 5.16 | 0.05 | 5.89 |
| 5.00E+01 | 1 | 5.16 | 0.1 | 6.09 |
| 2.50E+01 | 2 | 5.16 | 0.2 | 6.05 |
| 1.25E+01 | 4 | 5.16 | 0.4 | 5.91 |
| 1.00E+01 | 5 | 5.52 | 0.5 | 5.94 |
| 5.00E+00 | 10 | 5.53 | NA | NA |
| 2.50E+00 | 20 | 5.42 | NA | NA |
| | | | Symcel (50 CFU/sample) | |
| 1.00E+02 | | | 0.5 | 4.56 |

### INCORPORATION BY REFERENCE

All publications referenced herein are incorporated by reference in their entirety and for all purposes.

1. A method of interrogating a blood sample for microorganisms, comprising:
separating the blood sample into a first fraction and a second fraction under conditions that tend to concentrate microbes into the first fraction;
incubating the first and second fractions under conditions suitable for microbial growth; and
interrogating the first and second fractions for microbial growth, wherein
(a) the first fraction is interrogated using a first growth detection method for a first time interval and the second fraction is interrogated using a second growth detection method during a second time interval,
(b) the first growth detection method is more sensitive to microbial growth than the second detection method,
(c) the second time interval is longer than the first time interval, and
(d) if growth is not detected in the first fraction using the first growth detection method during the first time interval, the first fraction is interrogated for microbial growth for the remainder of the second time interval using a growth detection method other than the first growth detection method.

2. The method of claim 1, wherein the growth detection method other than the first growth detection method is the second growth detection method.

3. The method of claim 2, wherein the first growth detection method is calorimetry.

4. The method of claim 3, wherein the second growth detection method is pH, gaseous, or optical and, if optical, is selected from the group consisting of: optical measurement of turbidity; optical measurement of absorbance at one or more wavelengths, optical detection of a signal of a metabolic indicator dye; monitoring of autofluorescence, flow cytometry or any combination of the foregoing.

5. The method of claim 1, wherein the first and second fractions comprise microbial suspensions.

6. The method of claim 1, wherein the step of separating the blood sample into first and second fractions comprises centrifugation.

7. The method of claim 1, wherein the blood sample is incubated under conditions favorable for microbial growth prior to the step of separating the blood sample into a first fraction and a second fraction, and wherein the conditions favorable for microbial growth optionally comprise one or more of a temperature above ambient temperature, temperature of about 37 degrees centigrade, addition of a nutrient or a nutrient media, and/or addition of a material that adsorbs or inactivates an antimicrobial in the blood sample.

8. The method of claim 7, wherein microbial growth is not monitored during the incubation preceding the separation step.

9. A method of interrogating a blood sample for microorganisms, comprising:
contacting the blood sample with a resin capable of adsorbing antimicrobial agents;
performing one or more concentration steps to concentrate microorganisms into:
a) a pellet; and
b) a supernatant;
introducing a first subsample comprising at least a portion of the pellet into a calorimeter;
measuring heat flow from the first subsample, thereby monitoring growth of the first subsample; and
retaining a second subsample comprising a portion of the supernatant, wherein the second subsample is monitored for growth (i) by a method other than calorimetry, and/or (ii) over a time interval longer than an interval of monitoring the first subsample.

10. The method of claim 9, wherein if no growth is measured, the first subsample is removed from the calorimeter after a pre-determined period of time of about 0.5, 1, 2, 3, 4, 5 days.

11. The method of claim 9, wherein the second subsample is monitored for growth by optical, pH, gaseous, or impedance methods.

12. The method of claim 9, wherein the growth of at least one of the first and second subsamples is monitored based on an absolute signal.

13. The method of claim 9, wherein the growth of at least one of the first and second subsamples is monitored based on a relative signal.

14. The method of claim 9, wherein the second sample further comprises at least a portion of the pellet.

15. The method of claim 14 wherein the retained supernatant and remainder of the pellet are monitored for growth by optical, pH, gaseous, or impedance methods.

16. The method of claim 9, wherein the blood sample is incubated under conditions promoting microorganism growth prior to the concentration step.

17. The method of claim 9, wherein the calorimeter is a differential scanning or isothermal calorimeter.

18. The method of claim 9, wherein the first and second subsamples are monitored for growth in parallel for a first interval, and wherein if growth is not detected in the first sample during the first interval, the first subsample is removed from the calorimeter and is monitored for growth by a method other than calorimetry for a remainder of the time interval over which the second subsample is monitored for growth.

19. The method of claim 9, wherein the supernatant does not undergo substantial concentration or purification.

20. The method of claim 19, wherein all or substantially all of a volume of the supernatant is included in the second subsample.

21. The method of claim 19, wherein the blood sample is collected in a collection vessel, the concentration steps comprise centrifugation of the blood sample in the collection vessel, and the supernatant is aspirated or decanted from the collection vessel following centrifugation and optionally returned to the collection vessel following removal of the pellet.

22. The method of claim 21, wherein the step of retaining the second subsample comprises retaining the supernatant in the collection vessel.

23. The method of claim 9, wherein the blood sample is incubated under conditions favorable for microbial growth prior to the one or more concentration steps, and wherein the conditions favorable for microbial growth optionally comprise one or more of a temperature above ambient temperature, temperature of about 37 degrees centigrade, addition of a nutrient or a nutrient media, and/or addition of a material that adsorbs or inactivates an antimicrobial in the blood sample.

24. The method of claim 23, wherein microbial growth is not monitored during the incubation preceding the one or more concentration steps.

25. A method of detecting microbial growth in a blood sample, comprising the steps of:
causing one or more endothermic processes in the blood sample; and
detecting a heat flow from the sample, wherein the heat flow has a non-negative slope, thereby detecting microbial growth.

26. The method of claim 25, wherein the endothermic processes are caused by one or more of an anticoagulant and a lytic agent applied to the blood sample.

27. The method of claim 25, wherein the blood sample is pre-incubated under conditions that favor microbial growth prior to the step of detecting the heat flow from the sample.

28. The method of claim 25, wherein detection of heat flow comprises isothermal calorimetry.

29. The method of claim 25, wherein the endothermic processes comprise a micellization reaction.

30. The method of claim 25, wherein the step of causing the endothermic process includes contacting the blood sample with a lytic reagent, optionally saponin.

31. The method of claim 25, wherein the step of causing the endothermic process includes contacting the blood sample with an anticoagulant, optionally sodium polyanethole sulfonate.
